(19) United States
(12) Patent Application Publication
(10) Pub. No.: US 2020/0048646 A1
(43) Pub. Date: Feb. 13, 2020

(54) GENE EDITING AND TRANSGENE FREE MUTANT PLANTS

(71) Applicant: The Regents of the University of California, Oakland, CA (US)

(72) Inventor: Yunde Zhao, San Diego, CA (US)

(21) Appl. No.: 16/490,286

(22) PCT Filed: Mar. 7, 2018
(86) PCT No.: PCT/US2018/021280

§ 371 Date: Aug. 30, 2019

Related U.S. Application Data
(60) Provisional application No. 62/468,301, filed on Mar. 7, 2017.

(51) Int. Cl. C12N 15/82; C12N 9/22

(52) U.S. Cl. CPC C12N 15/8212; C12N 2310/20; C12N 9/22

(57) ABSTRACT

The disclosure provides compositions and methods to transform and edit the genome of plants and to measure heritable genetic modifications.

### CROSS REFERENCE TO RELATED APPLICATIONS

This application claims priority to U.S. Provisional Application Ser. No. 62/468,301, filed Mar. 7, 2017, the disclosures of which are incorporated herein by reference.

### STATEMENT REGARDING FEDERALLY SPONSORED RESEARCH

This invention was made with Government support under Grant No. R01GM114660, awarded by the National Institutes of Health. The Government has certain rights in the invention.

### TECHNICAL FIELD

The disclosure provides methods and compositions for gene editing in plants.

### INCORPORATION BY REFERENCE OF SEQUENCE LISTING

Accompanying this filing is a Sequence Listing entitled “Sequence_ST25.txt”, created on Mar. 7, 2018 and having 120,624 bytes of data, machine formatted on IBM-PC, MS-Windows operating system. The sequence listing is hereby incorporated herein by reference in its entirety for all purposes.

### BACKGROUND

The advancement of CRISPR/Cas9 genome editing technology offers unprecedented tools for precisely editing DNA sequences in Arabidopsis and in other organisms (Cong et al., 2013; Feng et al., 2013; Mali et al., 2013; Feng et al., 2014; Gao and Zhao, 2014). Genome editing by CRISPR/Cas9 has only three requirements: expression of the Cas9 protein, production of a guide RNA (gRNA) that complements the DNA sequences of the target gene, and the existence of an NGG Protospacer Adjacent Motif (PAM) site in the target sequence (Cong et al., 2013; Mali et al., 2013). Cas9 is recruited to the target DNA by the gRNA molecule, which targets a specific DNA sequence by base pairing. Once at the target site, the nuclease activities of Cas9 generate a double-strand break (DSB) a few base pairs upstream the PAM site. Small deletions or insertions in the target site are generated when the DSB is repaired by error-prone Non-Homologous End Joining (NHEJ) DNA repair. Because of its simplicity, CRISPR/Cas9 has been widely adopted by many laboratories. Several groups have developed CRISPR vectors for editing genes in Arabidopsis (Feng et al., 2013; Mao et al., 2013; Fauser et al., 2014; Feng et al., 2014; Gao and Zhao, 2014; Jiang et al., 2014; Li et al., 2014; Xing et al., 2014; Lowder et al., 2015; Ma et al., 2015; Zhang et al., 2015). Successful editing events in Arabidopsis have been widely reported. It is evident that CRISPR/Cas9 mediated gene editing technology can successfully produce various heritable mutations in Arabidopsis. However the majority of the reported analyses of the heredity of mutations generated by CRISPR/Cas9 did not segregate out the CRISPR/Cas9 construct. There are two major concerns about the existence of the Cas9/gRNA DNA in CRISPR alleles of Arabidopsis mutants. First, it is difficult to determine whether the mutation in T2 generation in a putative Arabidopsis mutant is actually inherited from the T1 generation or is newly produced by the Cas9/gRNA construct at T2 generation. It is essentially impossible to distinguish the two possibilities if the mutation is heterozygous. This point is extremely important because the newly produced mutation at T2 generation is likely somatic and not heritable. Second, the prolonged existence of the CRISPR/Cas9 construct in the mutants greatly increases the risk of producing off-target mutations.

### SUMMARY

Effective isolation of targeted mutations generated by CRISPR/Cas9 requires not only reasonable editing efficiency, but also an easy method to screen for the mutations. Editing events generated by CRISPR/Cas9 are normally identified by restriction enzyme digestion of PCR fragments or by in vitro digestion using purified Cas9 protein. Both methods are time-consuming and laborious. Simplified screening methods are urgently needed.

The disclosure provides an effective strategy to reliably isolate Cas9-free T2 plants that contain stably heritable mutations in Arabidopsis. The disclosure uses a cassette that enables the expression of a visual marker (e.g., luminescent or fluorescent marker) gene under the control of a strong promoter inserted into the CRISPR/Cas9 vector. The visual marker cassette allows one to visually select Cas9-free plants at T2 generation.

The disclosure provides recombinantly engineered, non-naturally occurring gene editing system comprising one or more vectors comprising (a) at least one first regulatory element operable in a plant cell and operably linked to at least one nucleotide sequence encoding a CRISPR-Cas system guide RNA (gRNA) for targeting a target sequence in a plant, (b) a second regulatory element operable in a plant cell operably linked to a nucleotide sequence encoding a class 2 CRISPR-associated nuclease, and (c) a third regulatory element operable in a plant cell operably linked to a fluorescent reporter and optionally (d) a fourth regulatory element linked to an antibiotic resistance gene, wherein component (a) is located on the same or different vector than components (b) and (c), whereby the guide RNA targets the target sequence and the CRISPR-associated nuclease cleaves the DNA molecule, whereby expression of the at least one gene product is altered; and, wherein the CRISPR-associated nuclease and the guide RNA do not naturally occur together. In one embodiment, (a), (b), and/or (c) are operably linked to a terminator sequence functional in a plant cell. In one embodiment, the class 2 CRISPR-associated nuclease is Cas9. In one embodiment, the plant is Arabidopsis thaliana, Medicago truncatula, Solanum lycopersicum, Glycine max, Brachypodium distachyon, Oryza sativa, Sorghum bicolor, Zea mays, or Solanum tuberosum. In one embodiment, the gRNA sequence is flanked by ribozyme sequences (see, e.g., SEQ ID NO:22). In one embodiment, the at least one first regulatory element comprises a pol II promoter sequence. In one embodiment, the first regulatory element comprises a DNA-dependent RNA polymerase III (Pol III) promoter sequence. In one embodiment, the Pol III promoter sequence is derived from a monocot plant. In one embodiment, the Pol III promoter comprises a U3 or U6 promoter nucleotide sequence. In one embodiment, the at least one first regulatory element is a UBQ10 promoter. In one embodiment, the at least one first regulatory element comprises at least two regulatory elements separated by a gRNA sequence. In one embodiment, the at least two regulatory element comprises a polIII promoter and a polII promoter. In one embodiment, the at least two regulatory elements comprise two pol III promoters. In one embodiment, the structure comprises U6 promoter—gRNA sequence—UBQ10 promoter—ribozyme—gRNA sequence—ribozyme. In any one or more of the prior embodiments, the nucleic acid construct further comprises a multiple cloning site (MCS) located between the promoter and the gRNA sequence or between the ribozyme and the gRNA sequence. In one embodiment, the second and/or third regulator element comprises a DNA-dependent RNA polymerase II (Pol II). In one embodiment, the nucleic acid construct further comprises a 15-30 bp long DNA sequence inserted into the MCS site of the nucleic acid construct, wherein said 15-30 bp long DNA sequence is complementary to the targeted sequence. In one embodiment, the fluorescent reporter is selected from the group consisting of GFP (green fluorescent protein), EGFP (enhanced green fluorescent protein), GFP_{UV} (UV-excited green fluorescent protein), RFP (red fluorescent protein), mRFP (modified red fluorescent protein), YFP (yellow fluorescent protein), mcherry, CFP (cyan fluorescent protein), mGFP (modified green fluorescent protein), ERFP (enhanced red fluorescent protein), BFP (blue fluorescent protein), EBFP (enhanced blue fluorescent protein), EYFP (enhanced yellow fluorescent protein) and ECFP (enhanced cyan fluorescent protein). In one embodiment, the fluorescent reporter is mCherry. In another embodiment, the system is designed to alter the expression of the at least one gene product that confers one or more of the following traits: herbicide tolerance, drought tolerance, male sterility, insect resistance, abiotic stress tolerance, modified fatty acid metabolism, modified carbohydrate metabolism, modified seed yield, modified oil percent, modified protein percent, and resistance to bacterial disease, fungal disease or viral disease.

The disclosure also provides a modified plant cell, seed or progeny produced by the recombinant engineered, non-naturally occurring gene editing system described in the preceding paragraph.

The disclosure also provide a plant comprising a plant cell containing a gene editing system of the disclosure.

In one embodiment, the disclosure provides a gRNA cassette or vector comprising a gRNA flanked by ribozymes (e.g., SEQ ID NO:22), wherein the ribozyme-gRNA-ribozyme construct is downstream and driven by a polIII promoter.

The disclosure also provides a method comprising (a) transforming a plant with a recombinant engineered, non-naturally occurring gene editing system of the disclosure, (b) selecting T1 plants by fluorescence or antibiotic resistance; (c) genotyping T1 plants to identify candidate plants and harvest seeds from individual plants; (d) visually screening for class 2 CRISPR-associated nuclease-free T2 seeds by measuring fluorescence, wherein no fluorescence is indicative of class 2 CRISPR-associated nuclease-free seeds; and (e) obtaining stable and heritable mutations from obtained T2 plants. In one embodiment, the class 2 CRISPR-associated nuclease is Cas9 and thus visually screening for Cas9-free seeds.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1 shows an annotation and restriction map of pHDE-35S-Cas9-mCherry.

FIG. 2 shows an annotation and restriction map of pHDE-35S-Cas9-U6-Ubiq.

FIG. 3A-B shows design a CRISPR/Cas9 vector to facilitate a visual screen for Cas9-free Arabidopsis seeds at T2 generation. (A) A schematic representation of the new CRISPR/Cas9 vector that contains a Cas9 expression cassette driven by the CaMV 35S promoter and an U6-promoter controlled gRNA production unit. More importantly, it also expresses mCherry from a strong promoter At2S3 in seeds. (B) A visual screen for T2 seeds that no longer harbor the CRISPR/Cas9 construct. The Cas9-free seeds do not produce the red fluorescence.

FIG. 4A-E show generation of abp1 mutants using the mCherry-containing CRISPR/Cas9 editing vector. (A) A schematic representation of the ABP1 gene and the sequences (SEQ ID NO:4 and 5) of the selected target sites for editing ABP1. PAM sites (NGG or CCN) are highlighted. CRP2 and CRP3 target opposite strands of the ABP1 genomic DNA. The restriction enzyme sites used for genotyping and screening for mutations are underlined. BsaJI recognizes CCNNGG while TaqI cuts TCGA. Note that Cas9 usually cuts 3 bp upstream of the PAM site. Therefore screening with BsaJI enzyme is not optimal. (B) Restrict digestion screen of T1 plants transformed with CRP2/CRISPR vector using the enzyme BsaJI. Plants with mutations generate PCR bands resistant to BsaJI digestion (arrow). Among the 15 samples shown here, four potentially have been edited at the ABP1 locus (#3, 5, 11, 14). (C) Restriction digestion of PCR products from T1 plants that have disrupted the TaqI site at the CRP3 target site. Note that sample #75 has very little WT DNA. The arrow points to TaqI resistant PCR band. (D) Three abp1 mutants with deletions/insertions at the CRP2 target site are shown. The wild-type (WT; SEQ ID NO:6) abp1-cod (SEQ ID NO:7) has a 4 bp deletion and the abp1-c3d (SEQ ID NO:8) has a 3 bp deletion. WT (SEQ ID NO:9) The abp1-c8i (SEQ ID NO:10) has a very complex mutation. (E) Two editing events at the CRP3 site (WT: SEQ ID NO:11) that resulted in two stable Cas9-free abp1 alleles. One has a 12 bp deletion (SEQ ID NO:12) and the other deletes 42 bp delection near the target site. Note that the 42 bp deletion is not shown in full in the figure.

FIG. 5A-E shows CRISPR/Cas9-mediated deletions of a large DNA fragment between two gRNA target sites in Arabidopsis. (A) CRISPR plasmids were produced that target three sites of the ABP1 gene. An RGR and CRP2 modules were combined to delete the first three exons. The RGR and CRP3 were also combined in another plasmid. RGR is controlled by the Ubiquitin 10 promoter (UBQ10). Boxes refer to ABP1 exons. Vertical arrows point to gRNA target sites. ABP1-U409 and ABP1-CRP2-GT2 are the primer pair used in the PCR screening. The RGR sequence and design are shown in FIG. 2. (B) PCR amplification using ABP1-U409 and ABP1-CRP2-GT2 primers and the genomic DNA from Cas9-free T2 plants generated from a single T1 plant transformed with the RGR-CRP2 dual-gRNA vector. About half of the plants contained a deletion. Note that this primer pair preferentially amplifies the small fragment and cannot differentiate homozygous from heterozygous plants. (C) A schematic description of the abp1-c2 mutation, which is a deletion of 1141 bp including the first three exons and 304 bp of the ABP1 promoter. The dashed line represents the deleted region. (D) Identification of a second abp1 allele that has a large deletion. Only 2 plants (#105 and #115) out of 96 Cas9-free T2 plants from a single RGR-CRP2 T1 plant contained a deletion (arrow). (E) Further sequencing analysis show that the deletion is 711 bp, which is the exact expected size generated by gRNAs targeting RGR and CRP2 sites.

FIG. 6A-B shows a general method for reliably isolating stable and heritable targeted mutants using CRISPR/Cas9 editing technology in Arabidopsis. (A) A flowchart for isolating CRISPR alleles of Arabidopsis mutants. The key is to use the visual screen to quickly identify Cas9-free T2 seeds. Mutations in Cas9-free T2 plants are stably transmitted to next generations following Mendelian genetics (Table II). (B) A schematic description of the mosaic nature of mutations generated by CRISPR/Cas9 in T1 plants. If a founder cell for a flower is mutated, the seeds generated from that particular flower will contain heritable mutations (blue or purple). However, seeds in the majority of the siliques do not contain heritable mutations. Red refers to seeds with mCherry-CRISPR/Cas9 construct.

FIG. 7 shows an annotated sequence of a vector of the disclosure (SEQ ID NO:13) and coding sequences (SEQ ID NO:14-16).

FIG. 8 shows an annotated sequence of a vector of the disclosure (SEQ ID NO:17) and coding sequences (SEQ ID NO:18-20).

FIG. 9 shows an examples of an RGR construct of the disclosure.

FIG. 10 shows a sequence of an RGR construct of the disclosure (SEQ ID NO:21).

### DETAILED DESCRIPTION

As used herein and in the appended claims, the singular forms “a,” “and,” and “the” include plural referents unless the context clearly dictates otherwise. Thus, for example, reference to “a polynucleotide” includes a plurality of such polynucleotides and reference to “the seed” includes reference to one or more seeds, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

CRISPR/Cas9 gene editing technology is a powerful tool for creating targeted mutations in Arabidopsis, but it is important to identify mutations in Cas9-free T2 plants to ensure that mutations observed can be stably transmitted to future generations. The fluorescence-based visual screen methods and compositions of the disclosure facilitates the isolation of Cas9-free T2 seeds easily and quickly. In addition, by combining with the use of dual gRNAs, the methods and composition reliably identifies useful targeted mutations in Arabidopsis.

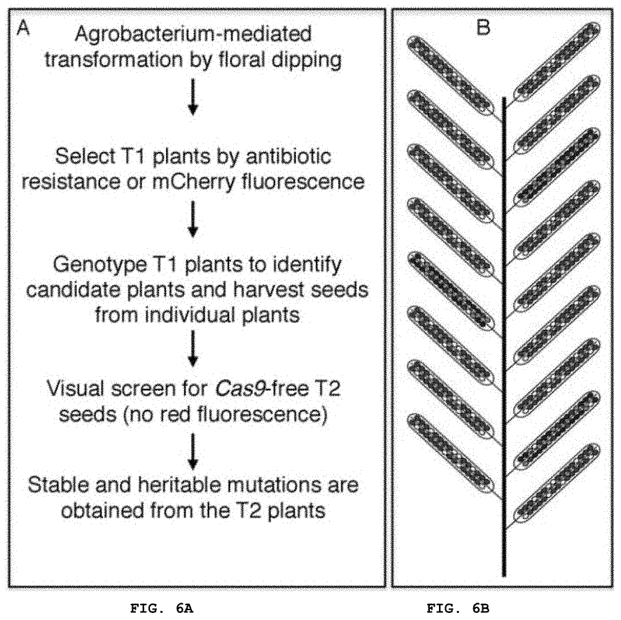

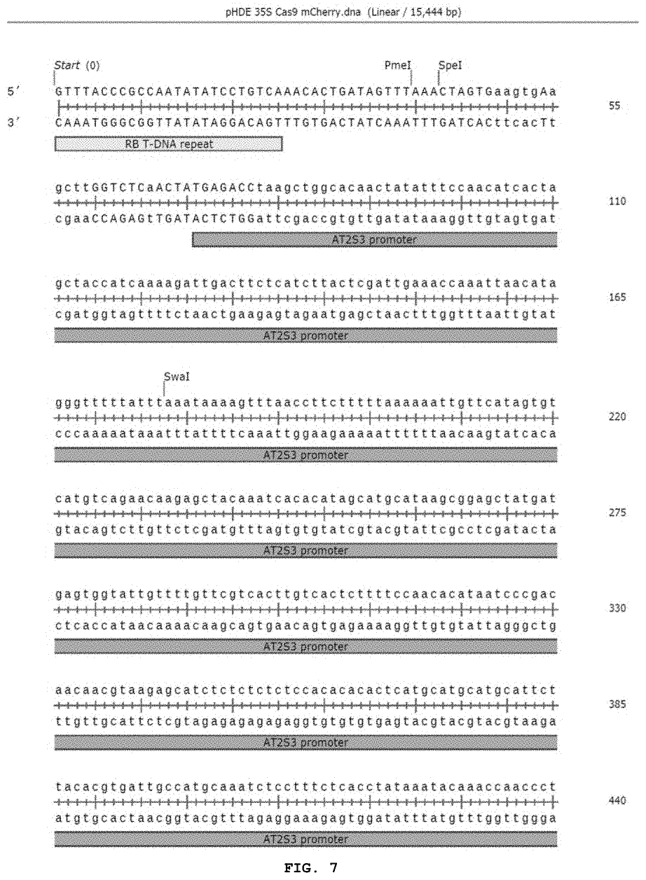

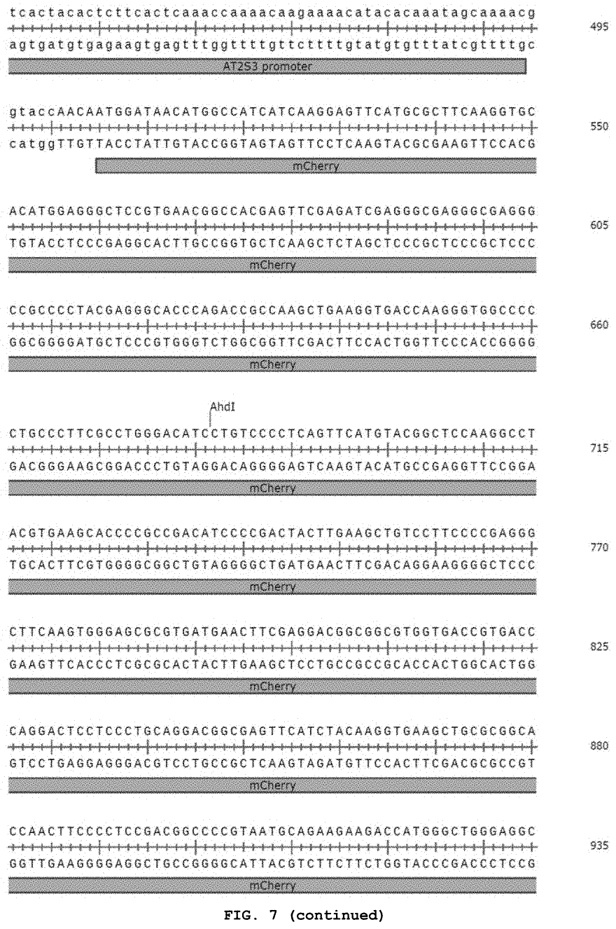

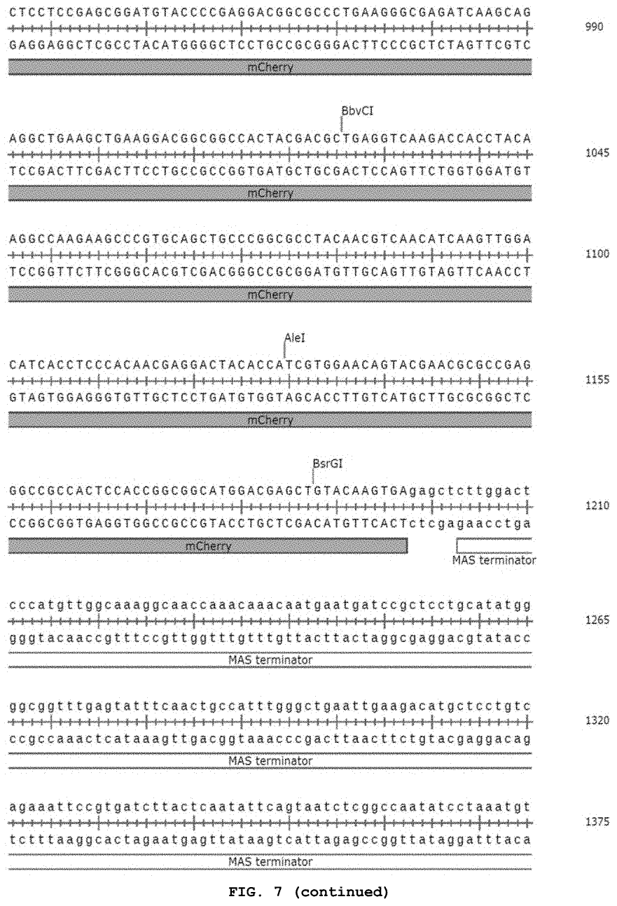

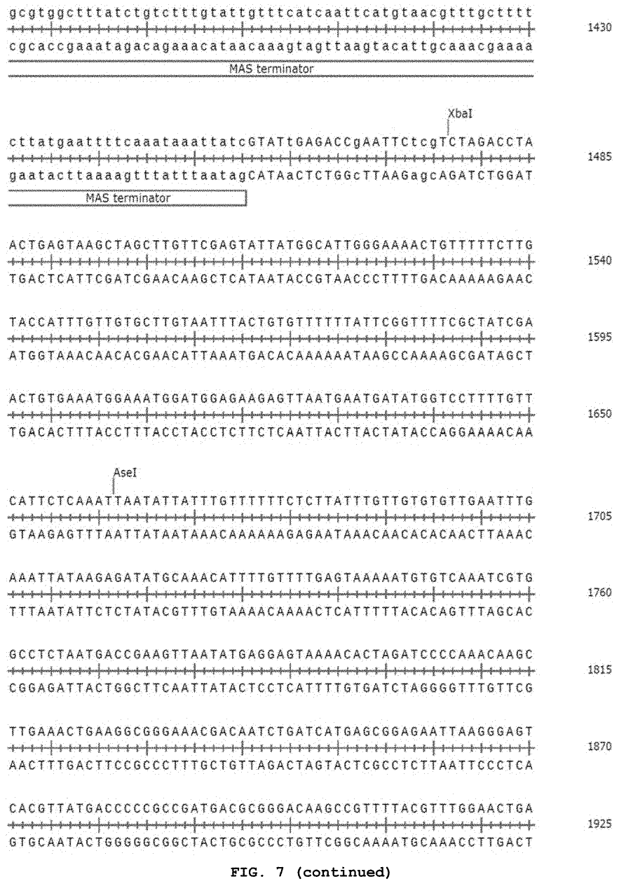

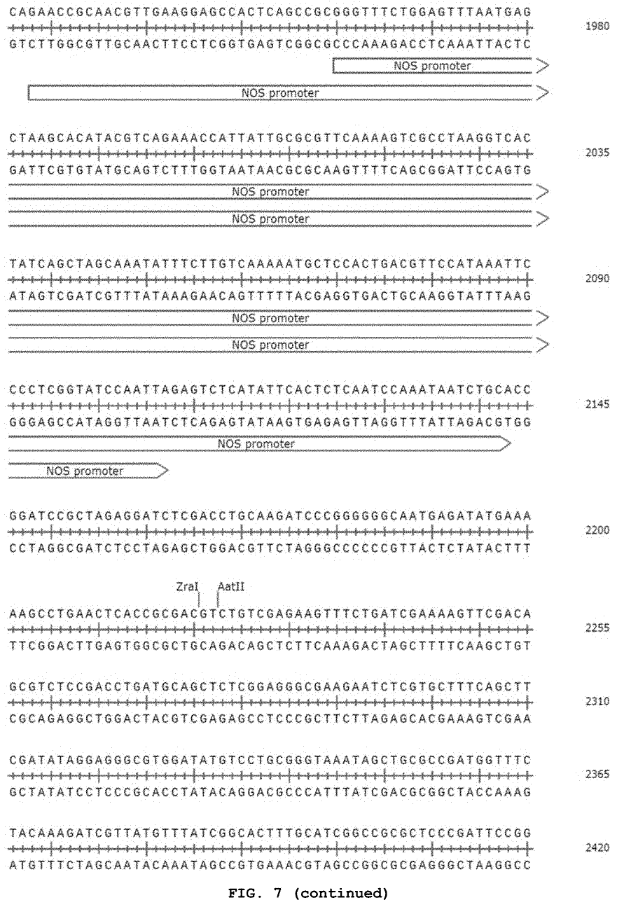

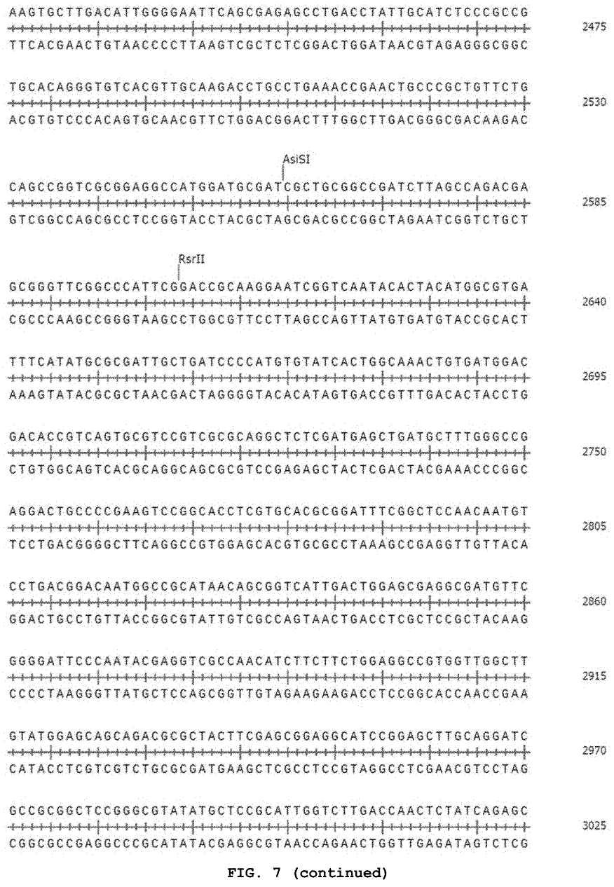

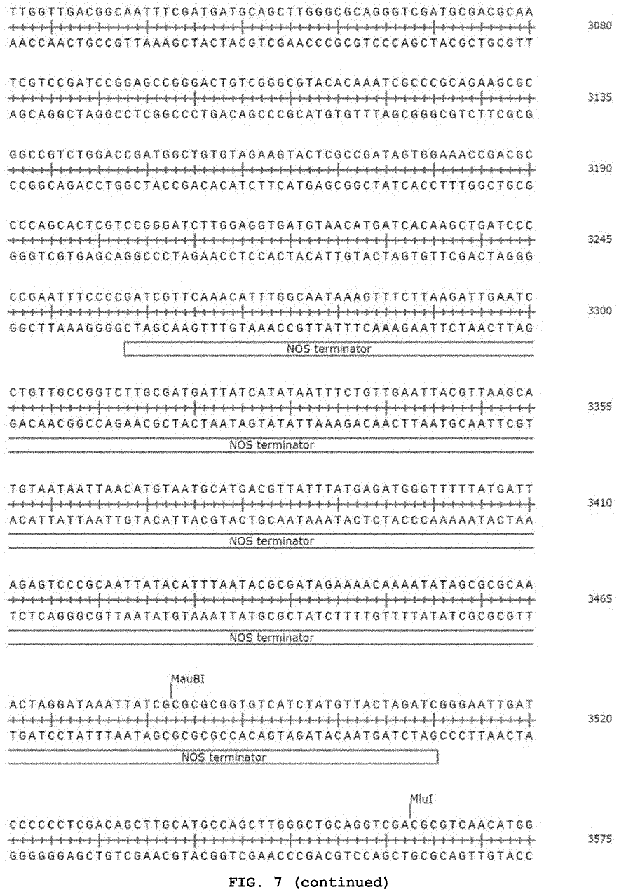

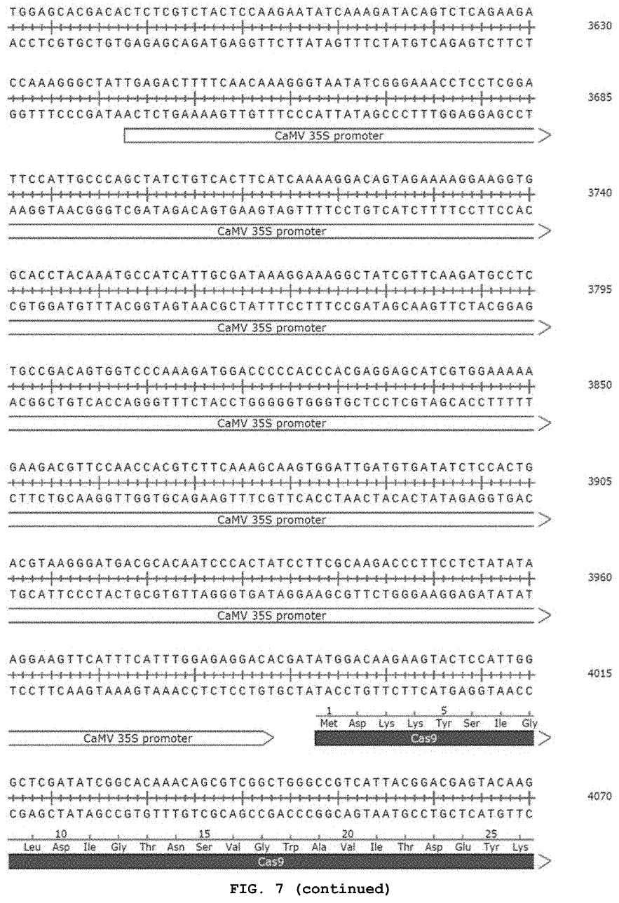

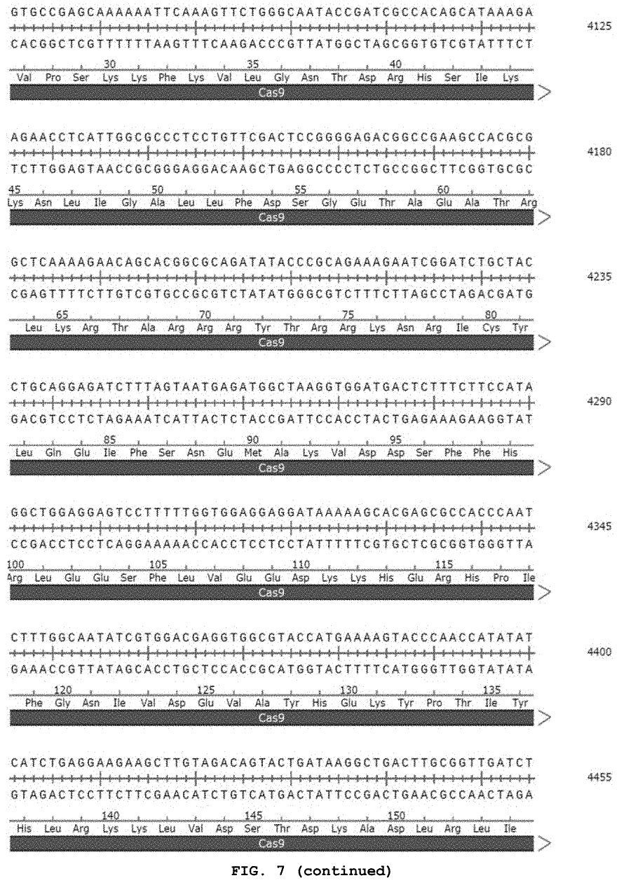

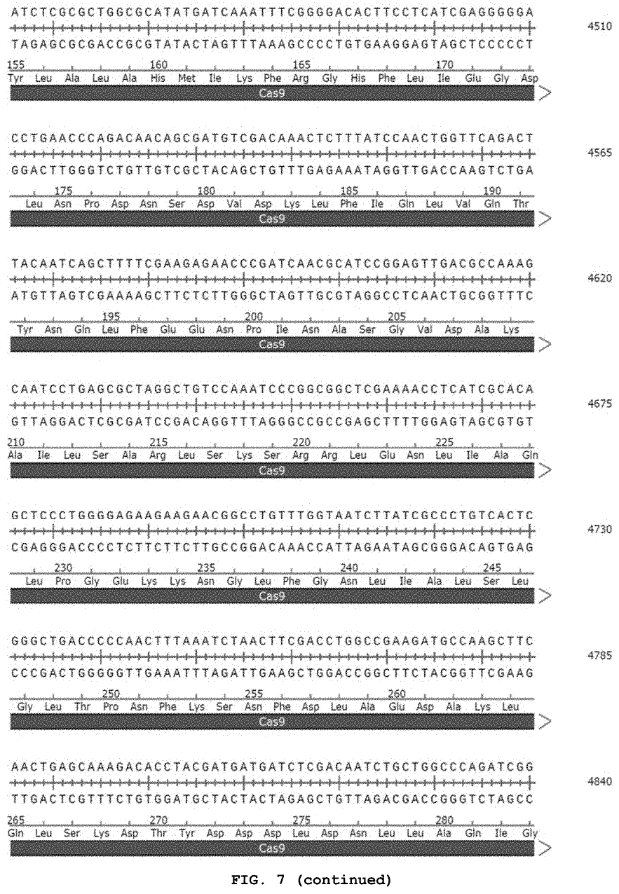

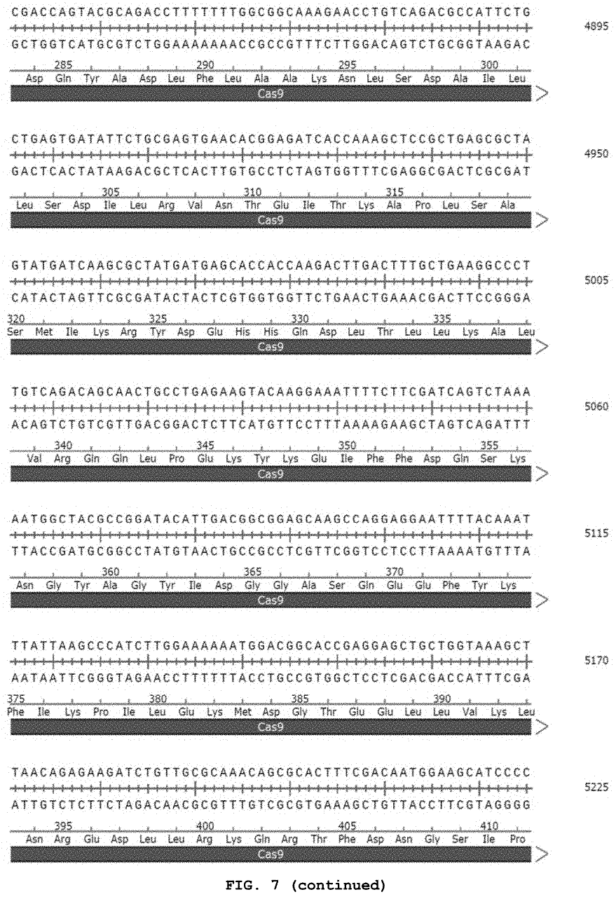

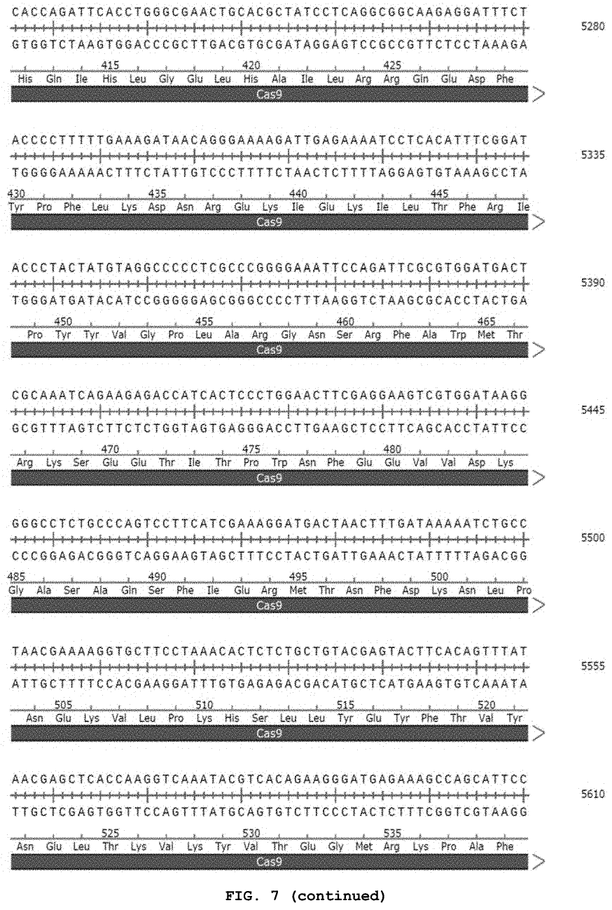

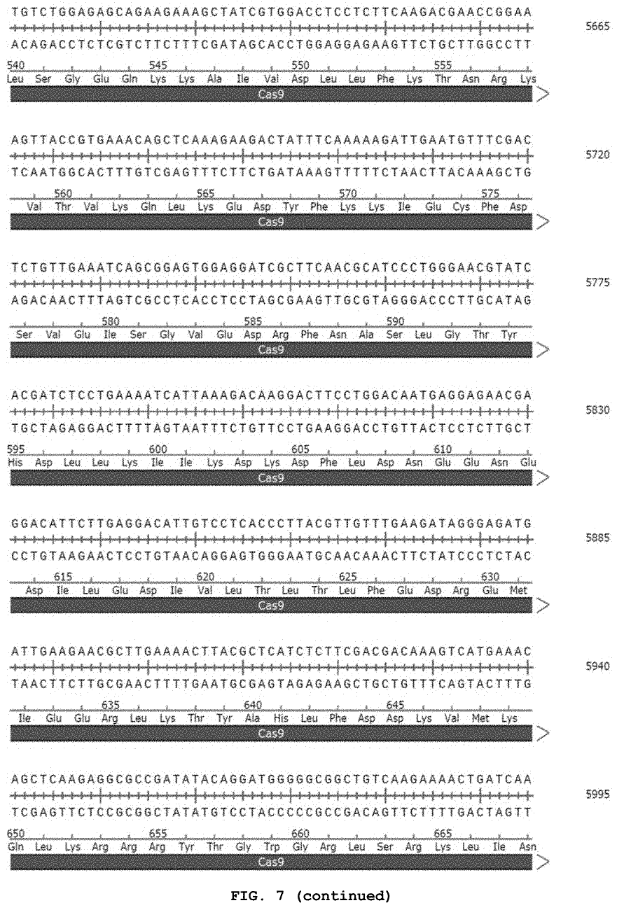

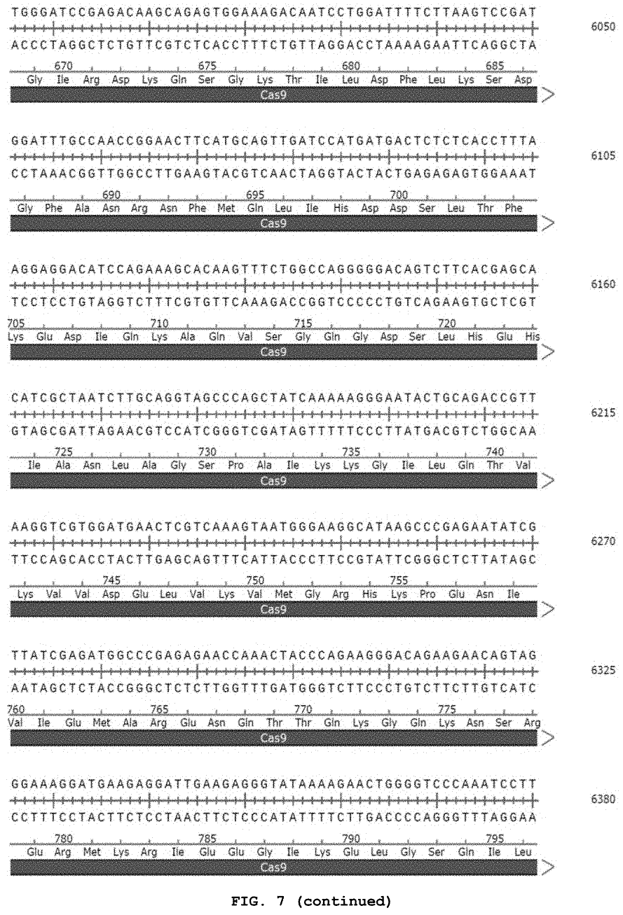

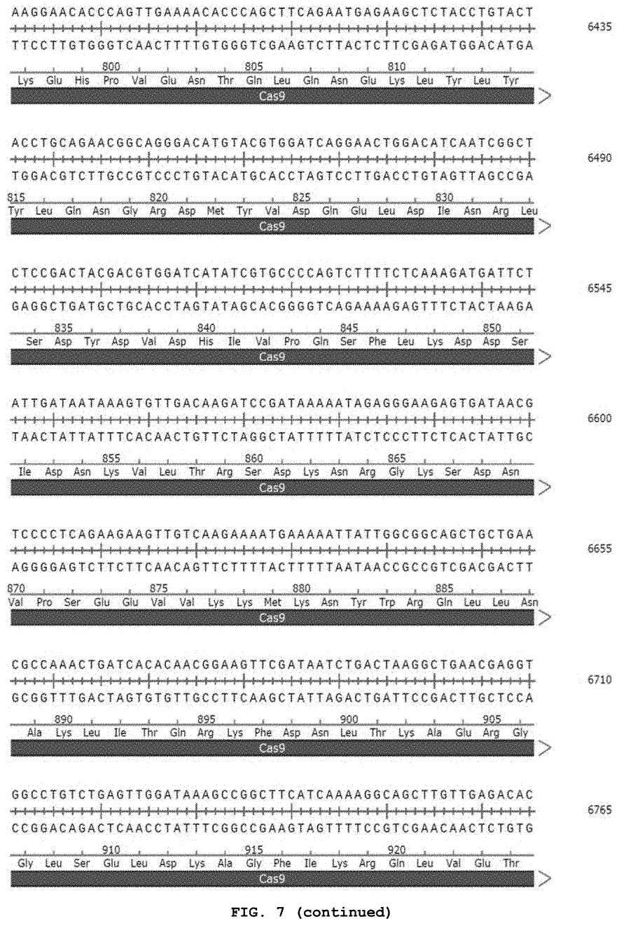

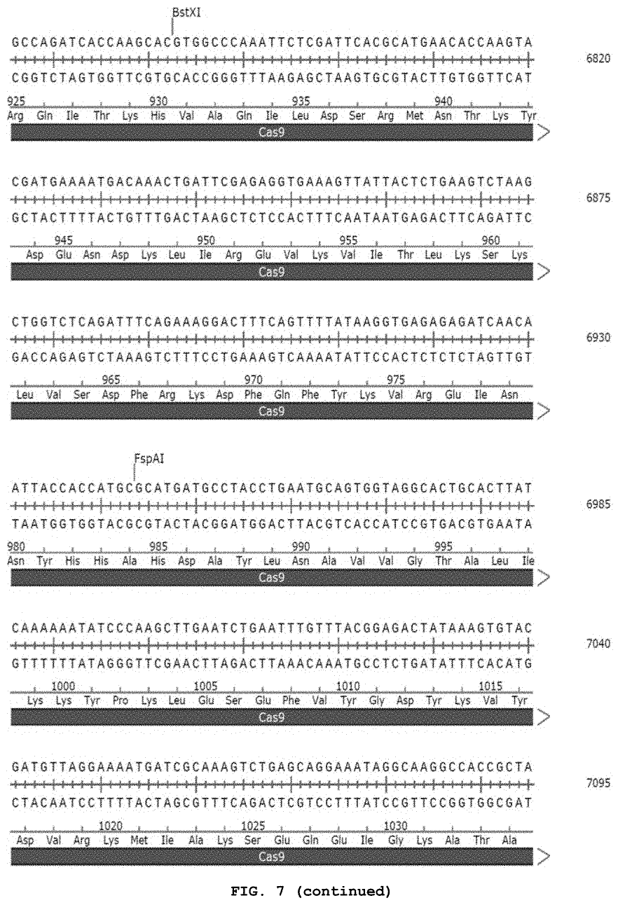

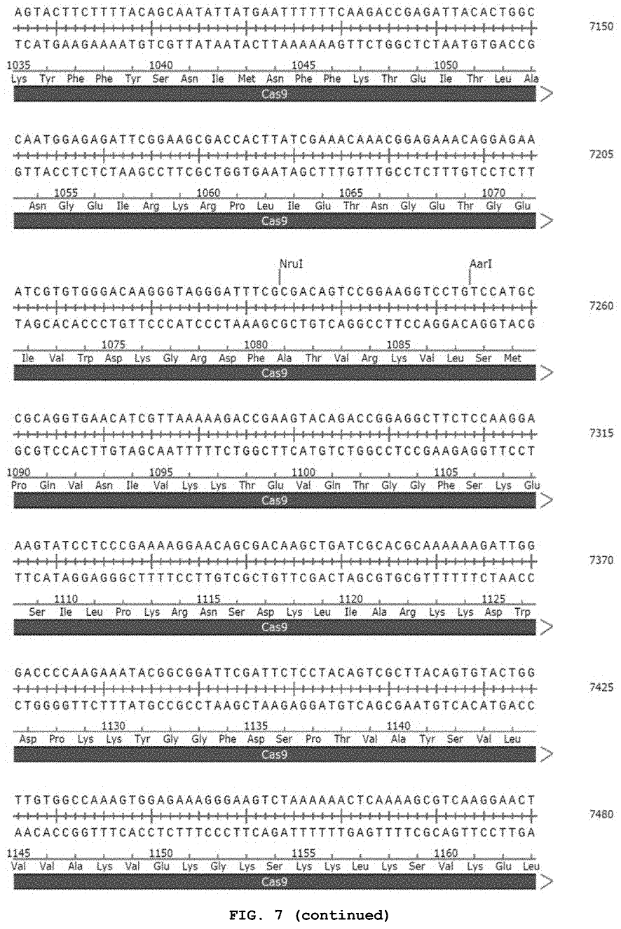

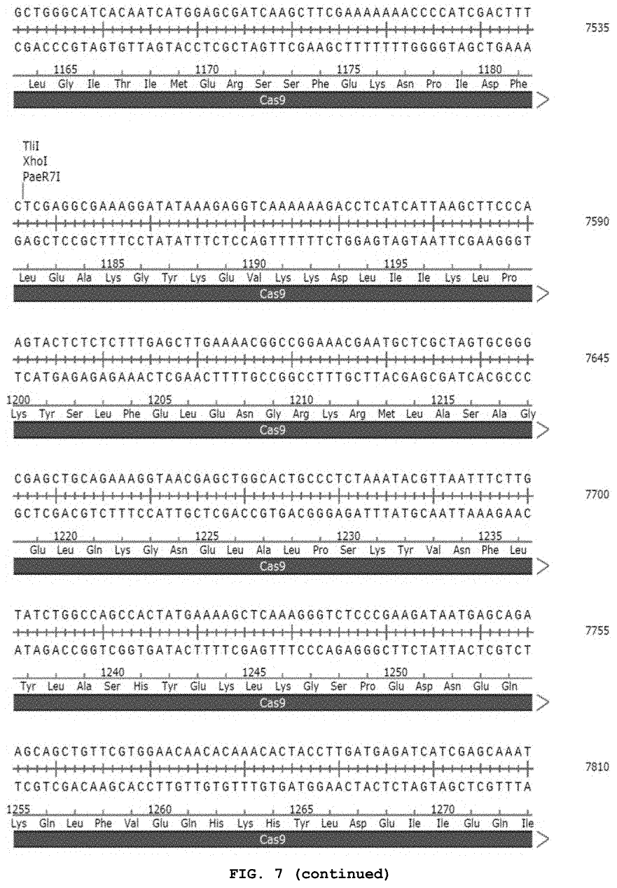

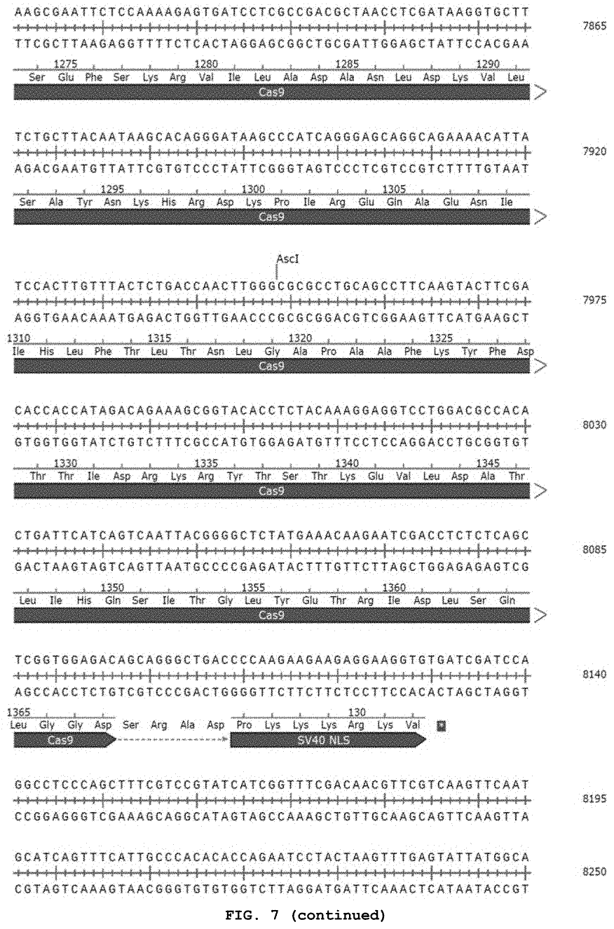

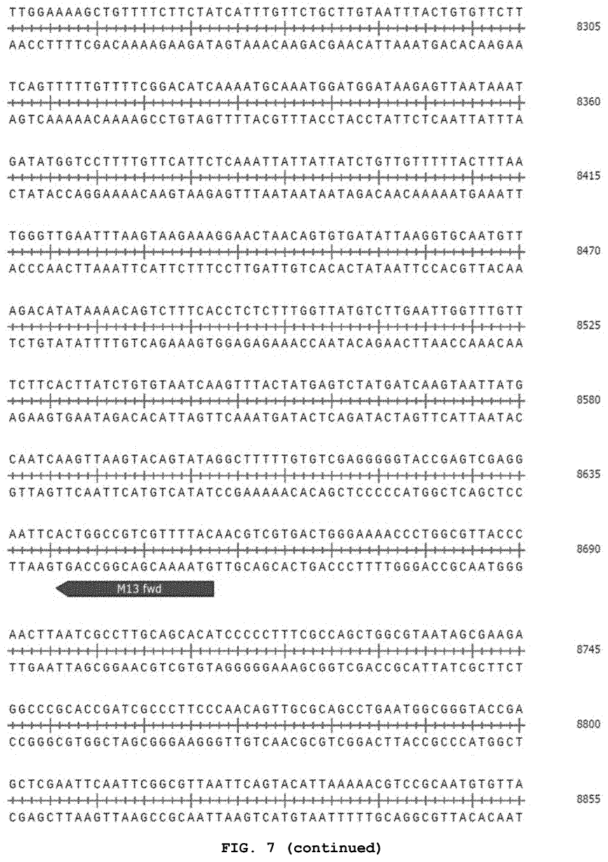

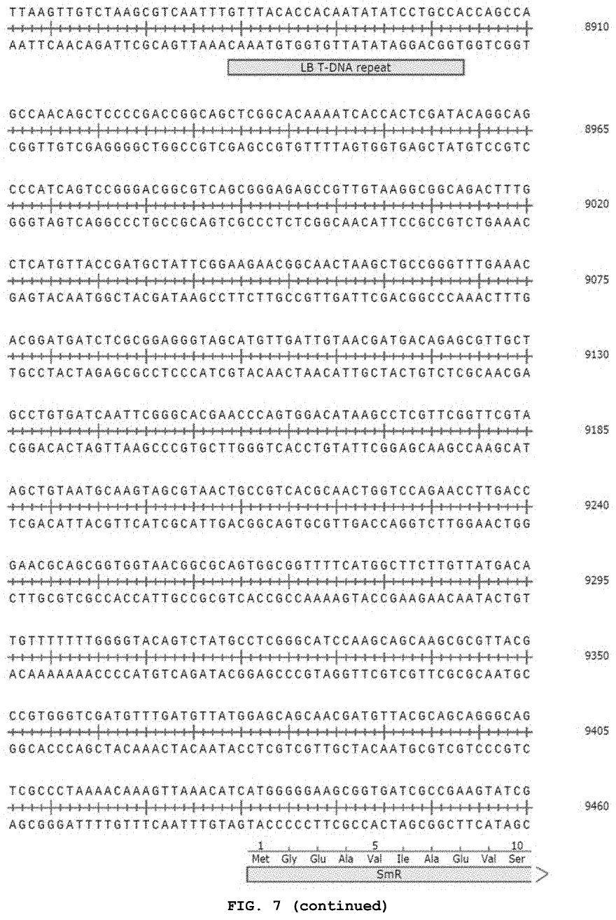

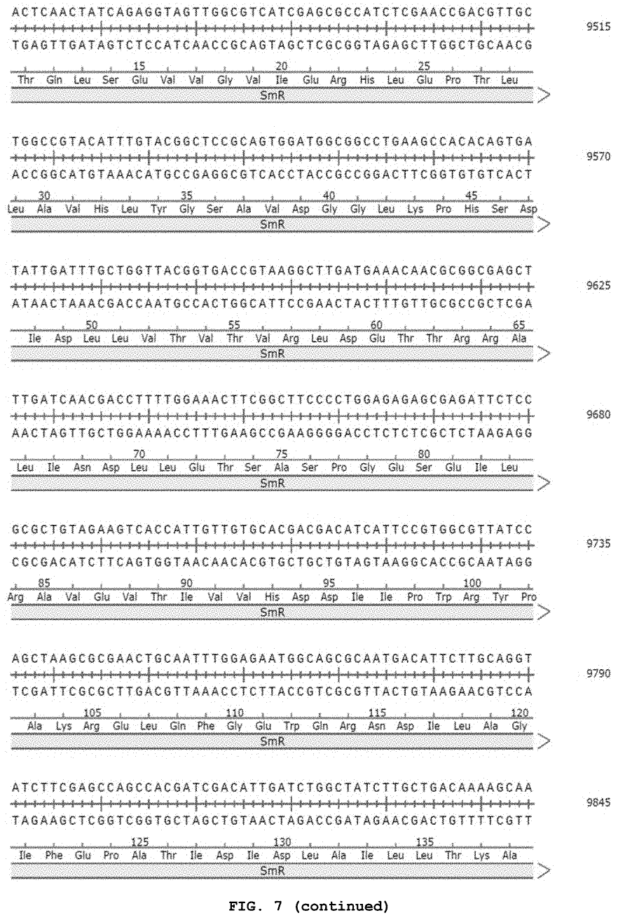

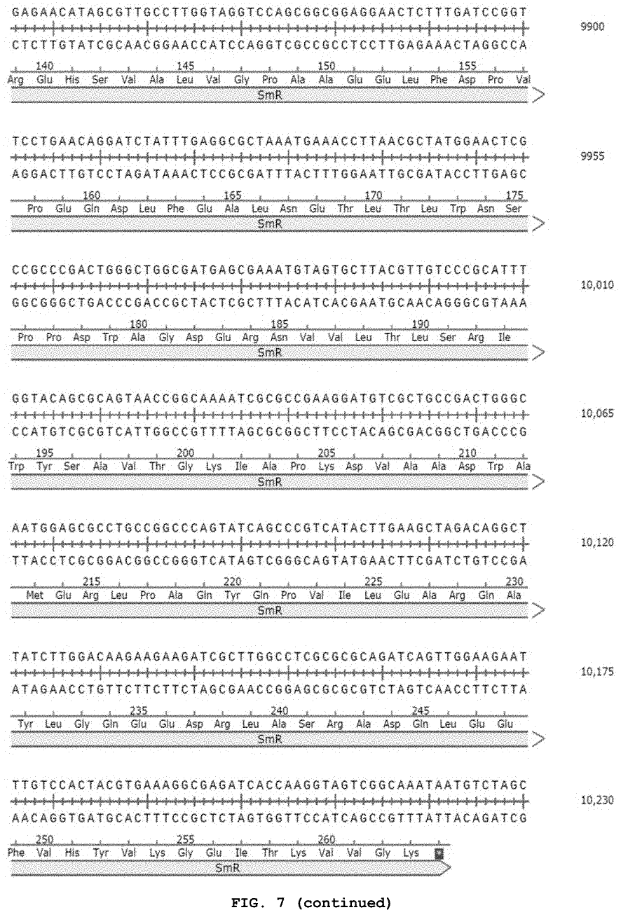

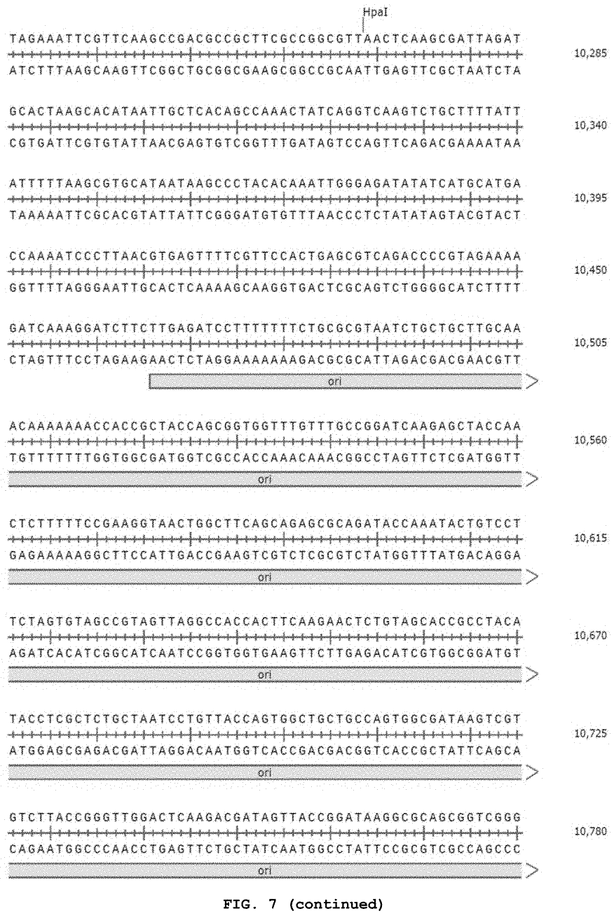

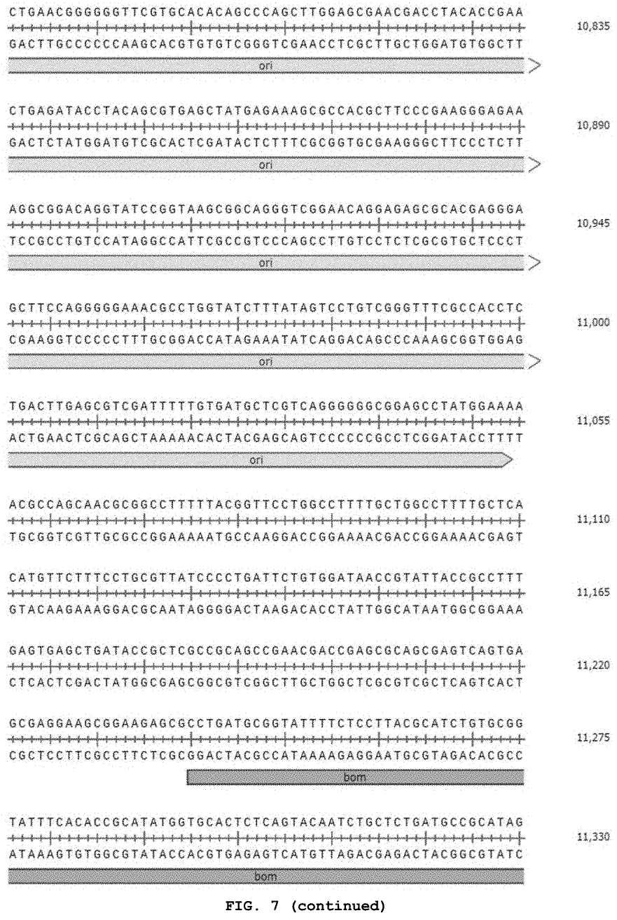

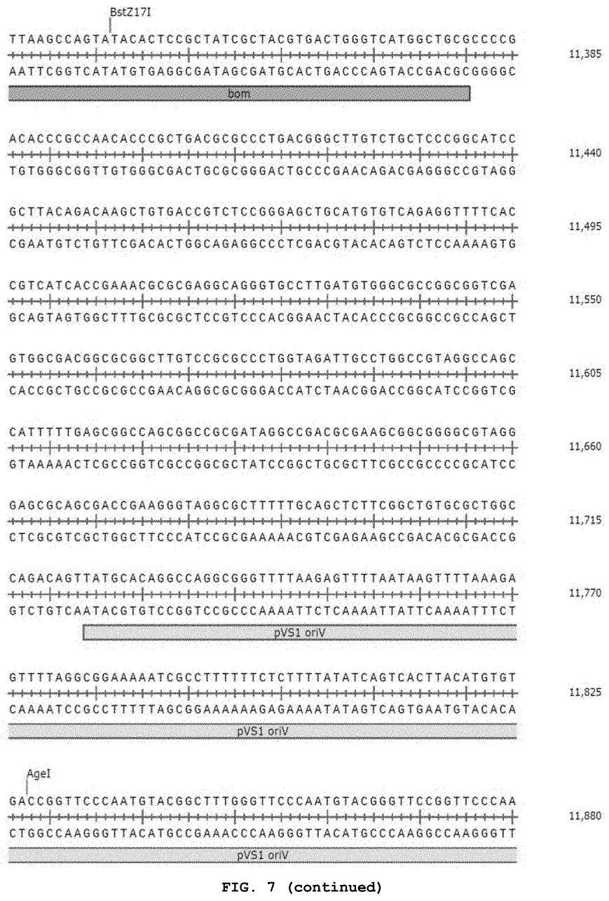

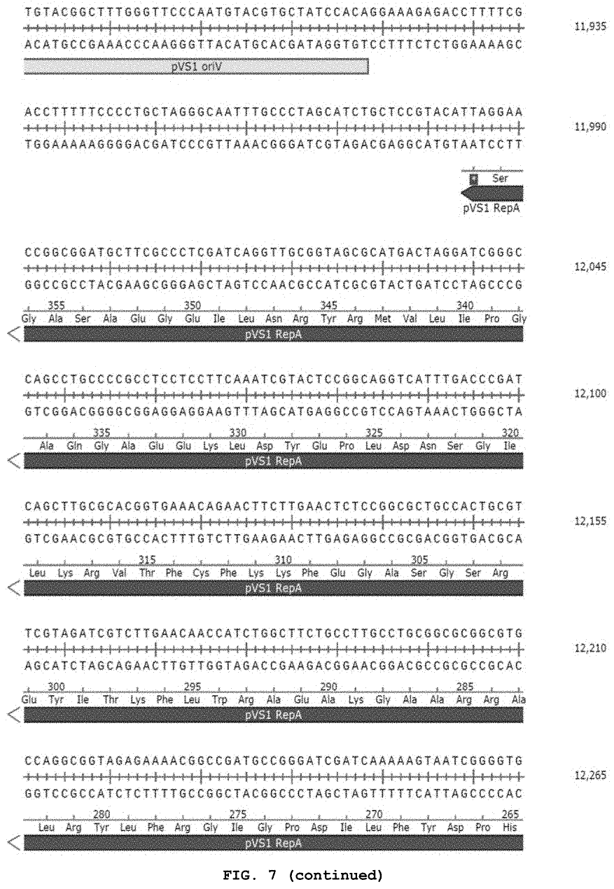

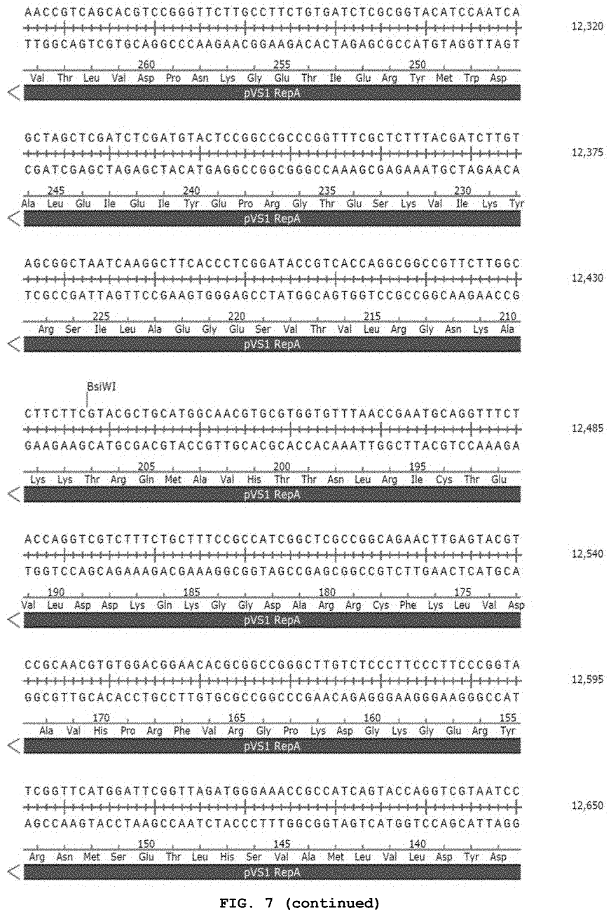

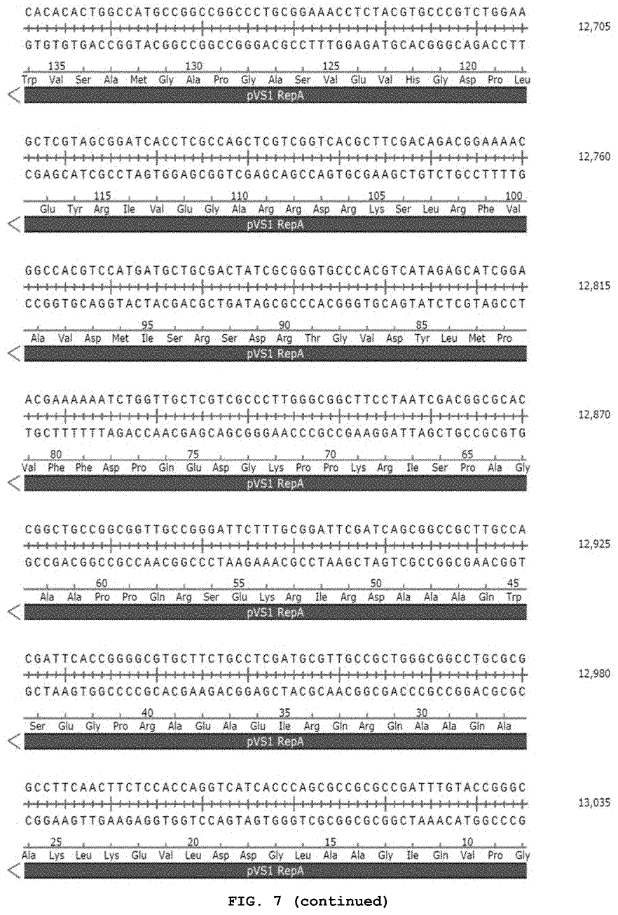

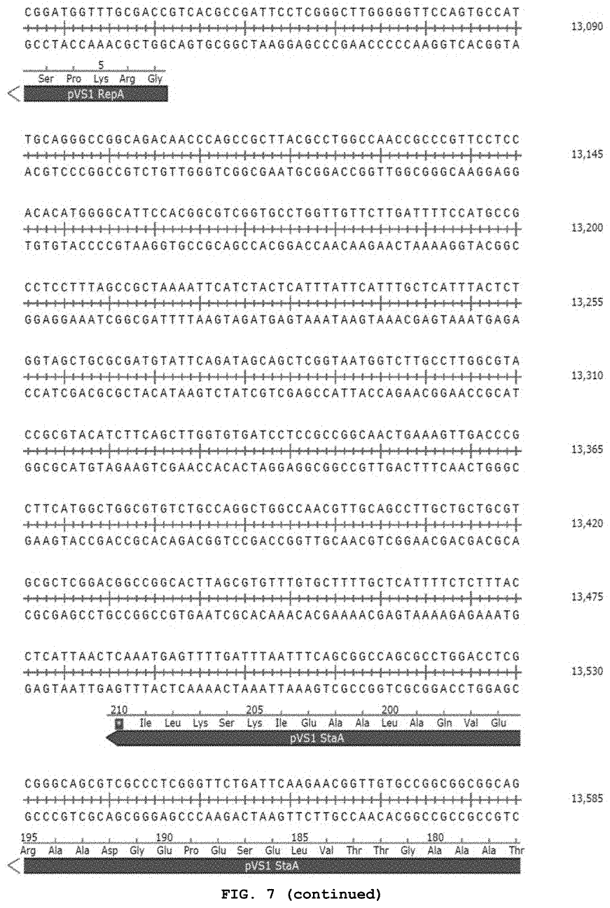

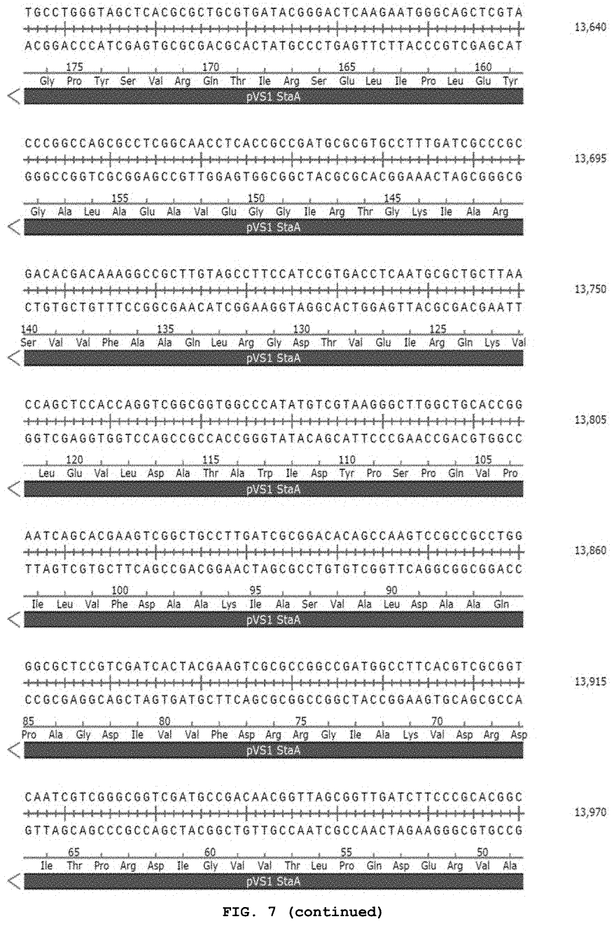

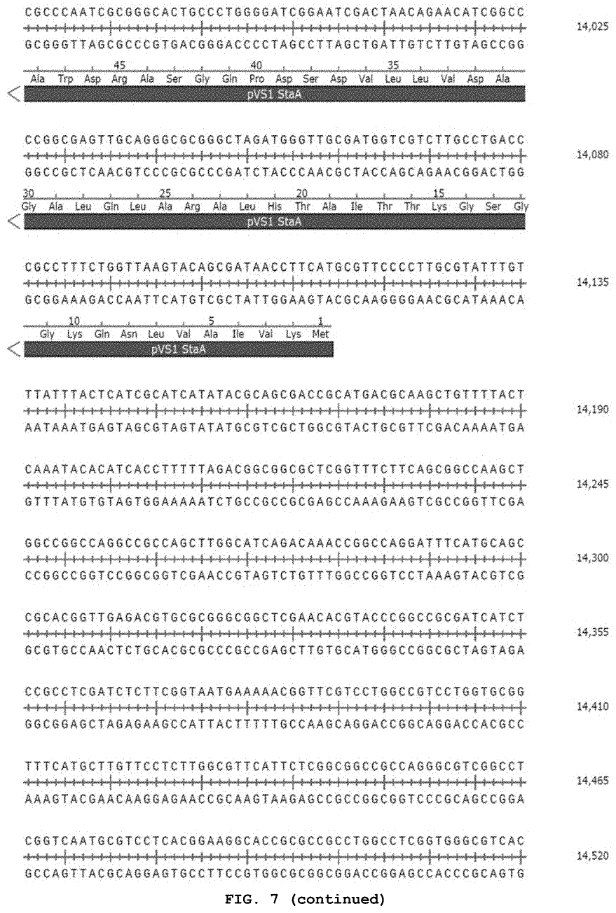

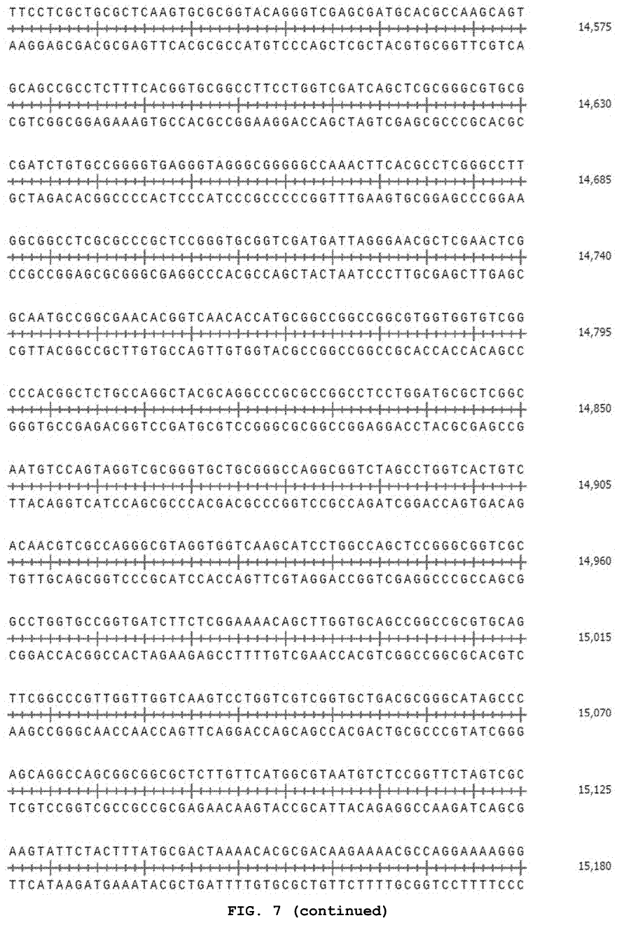

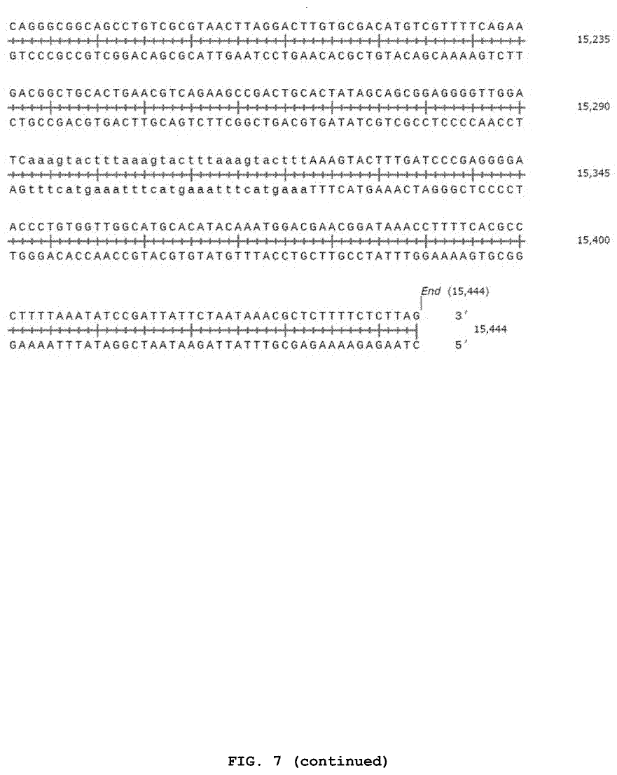

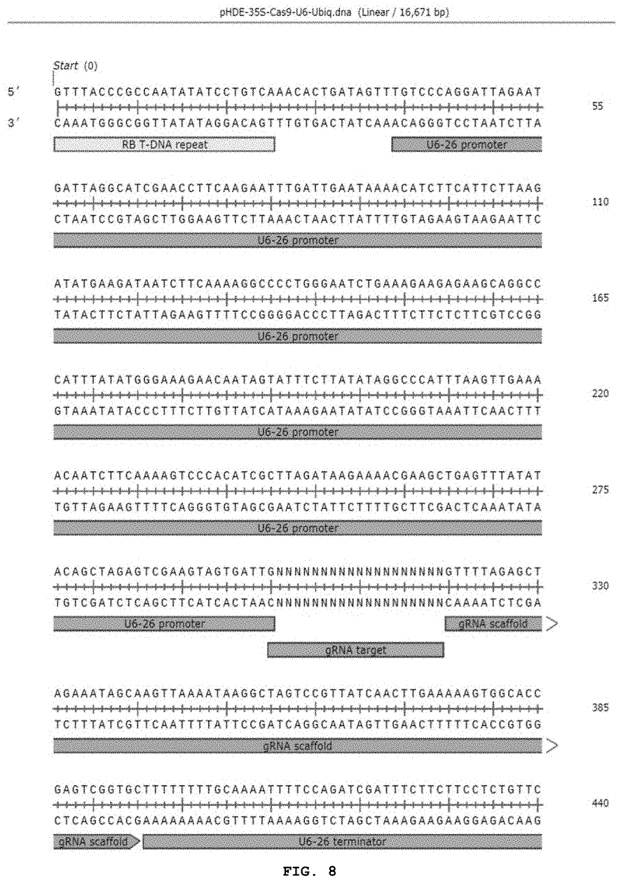

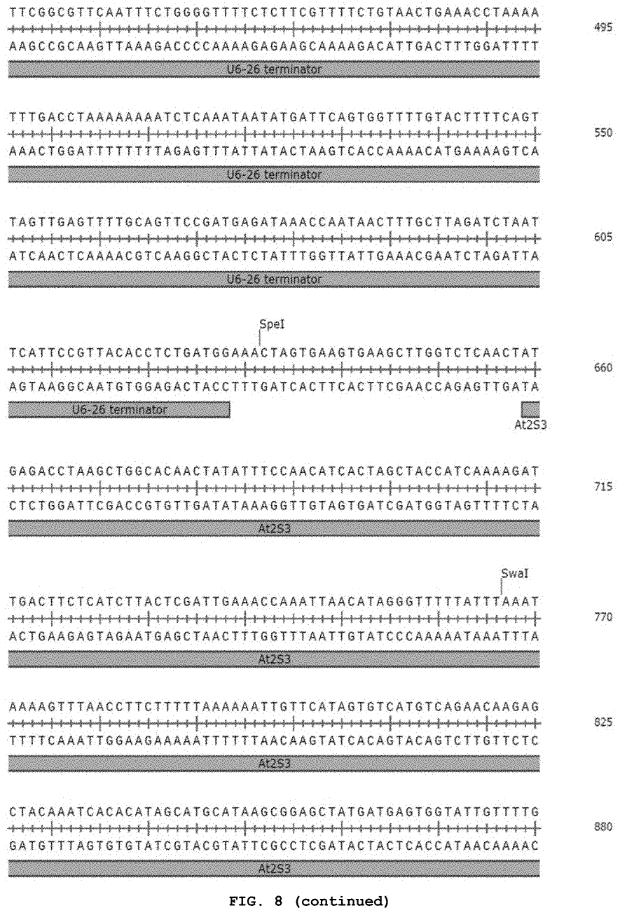

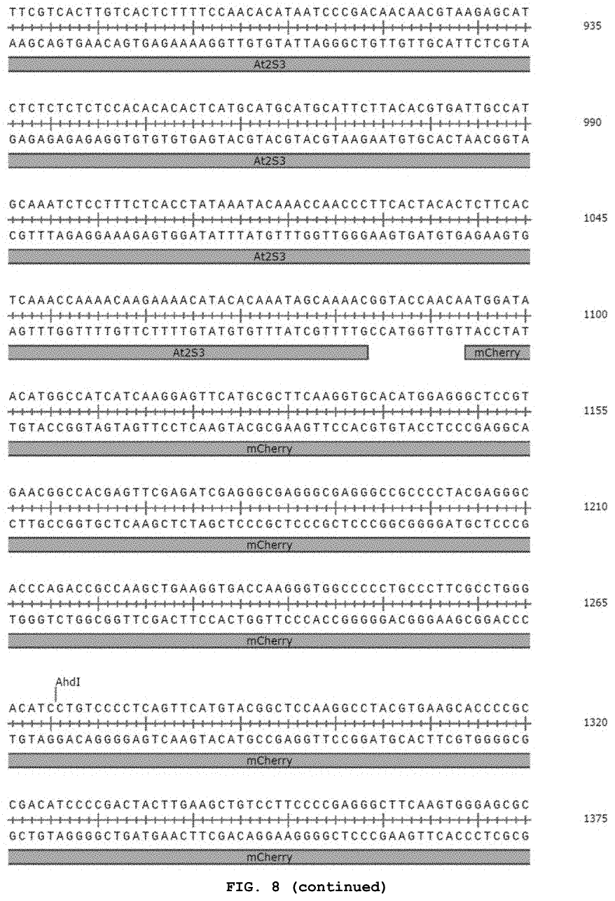

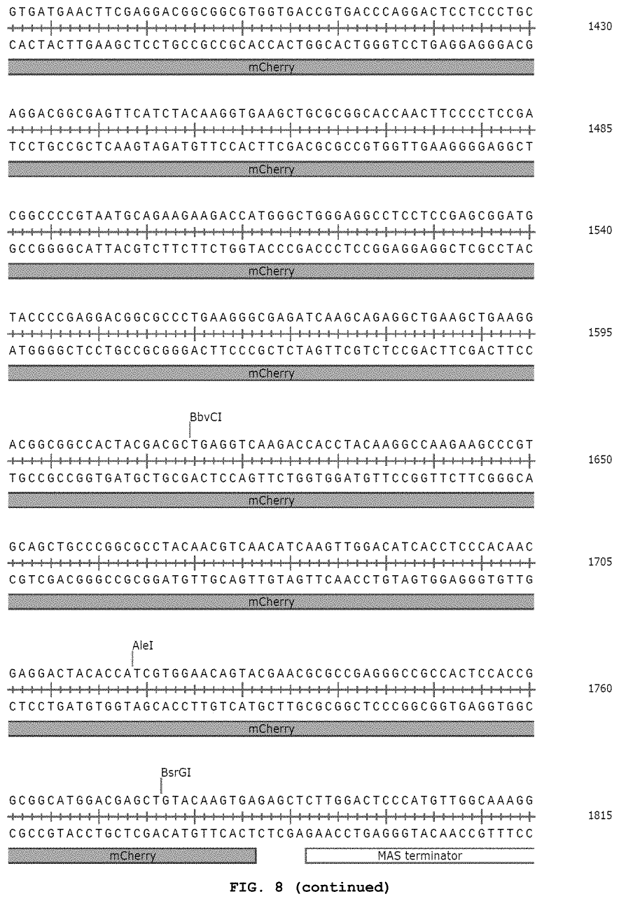

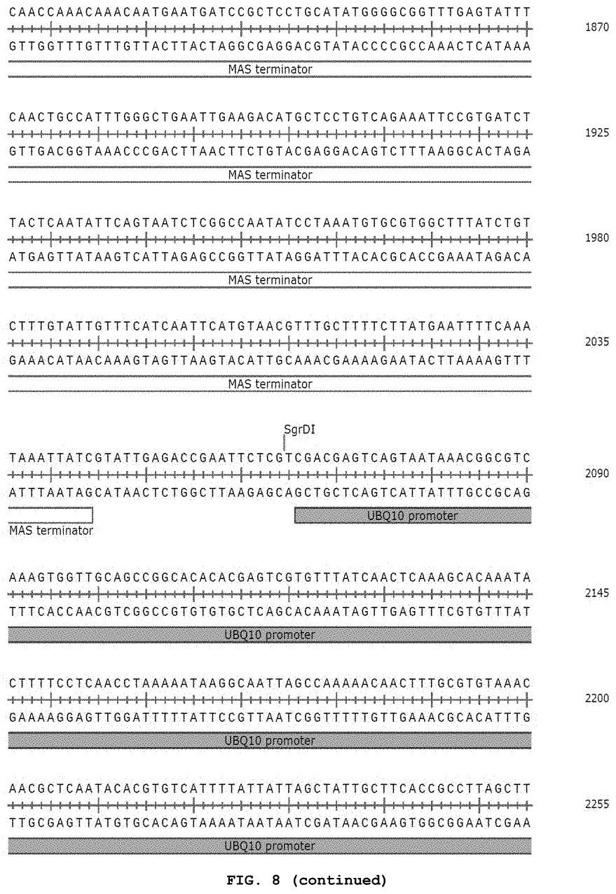

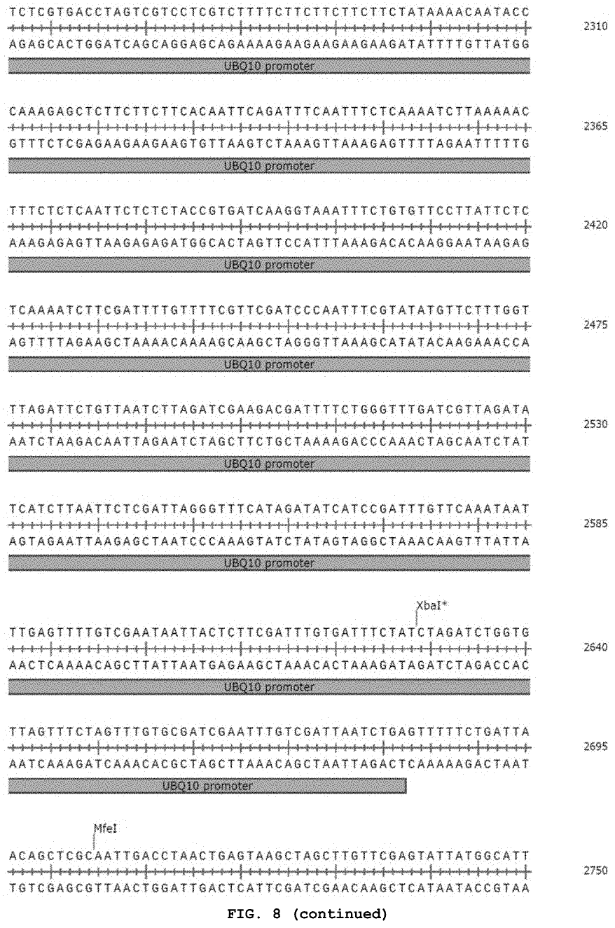

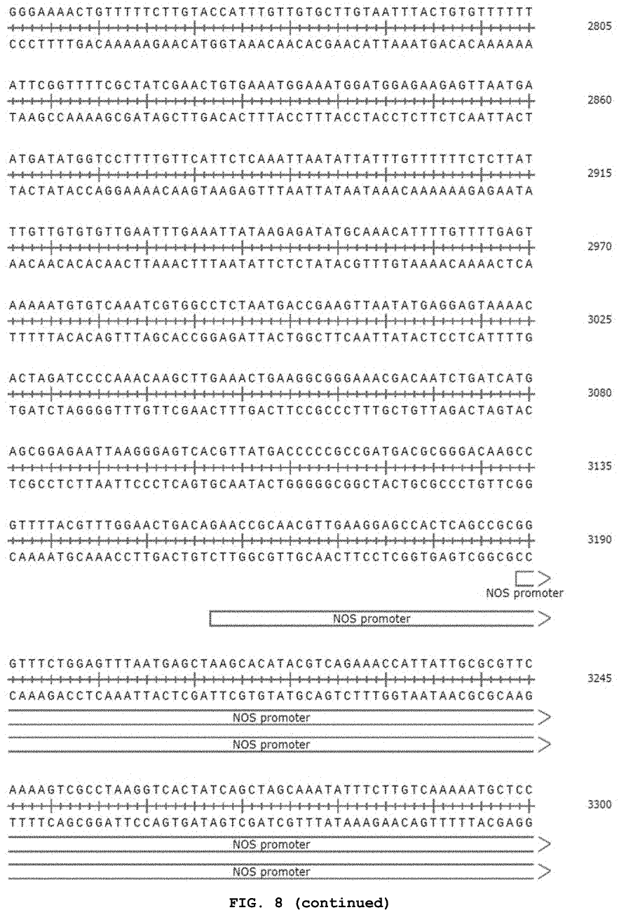

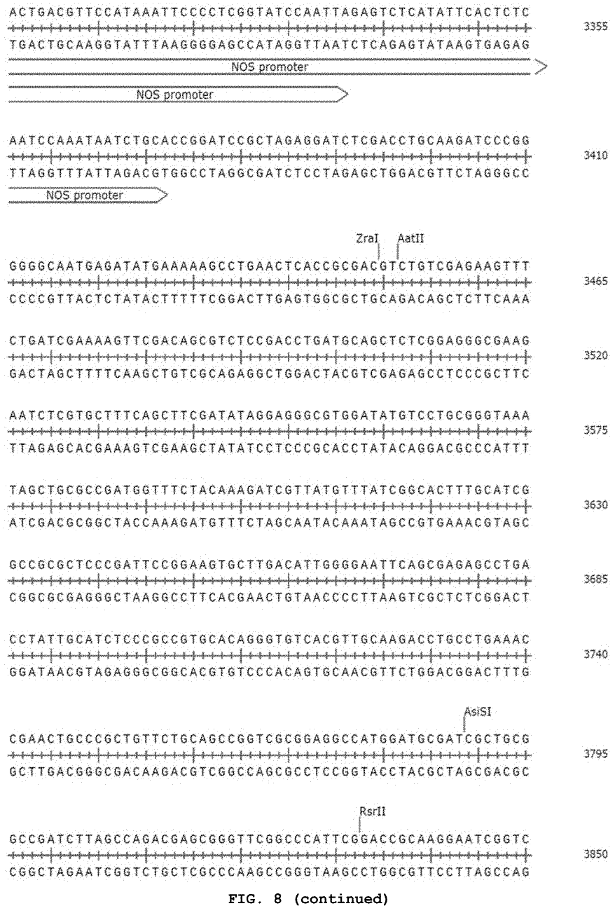

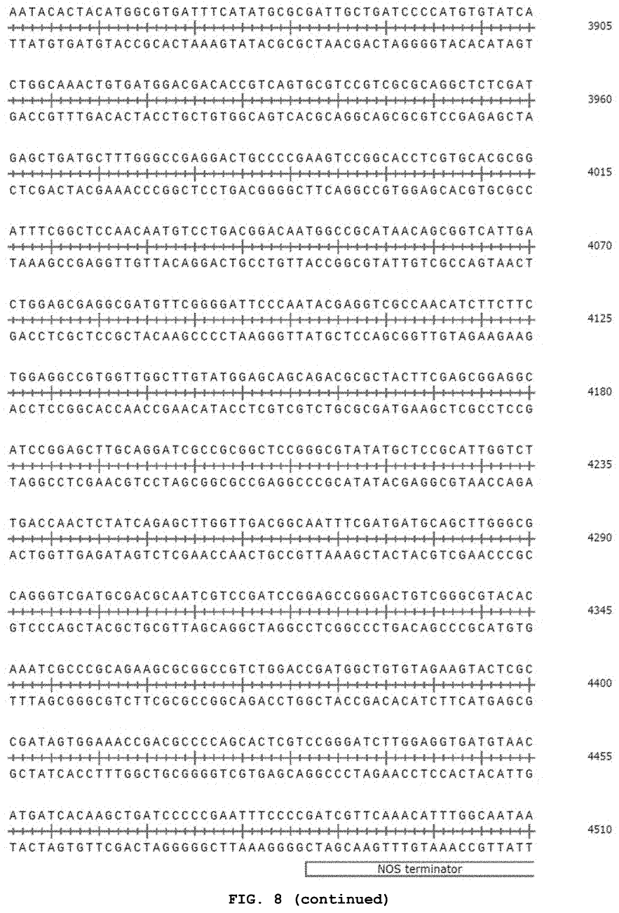

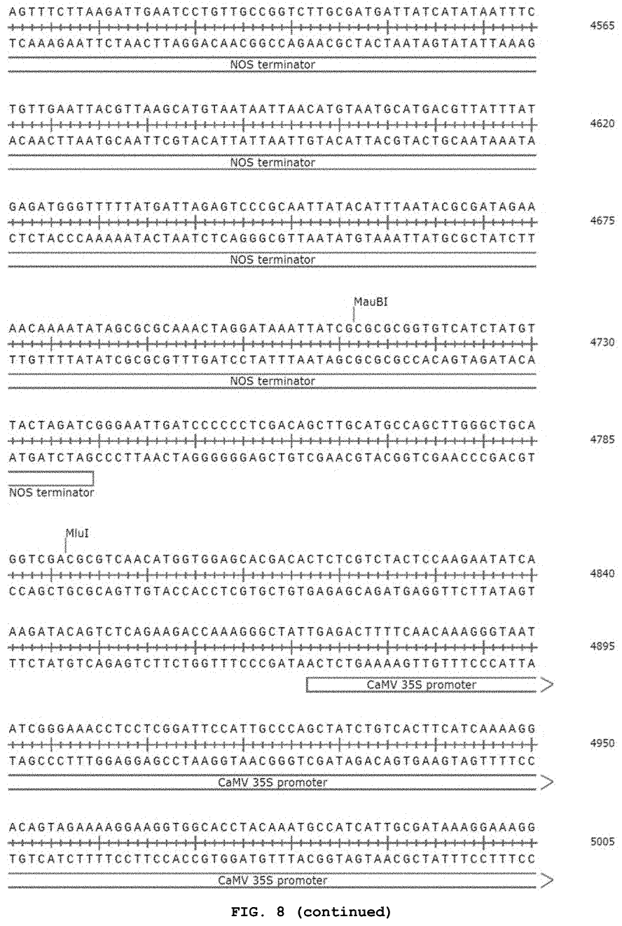

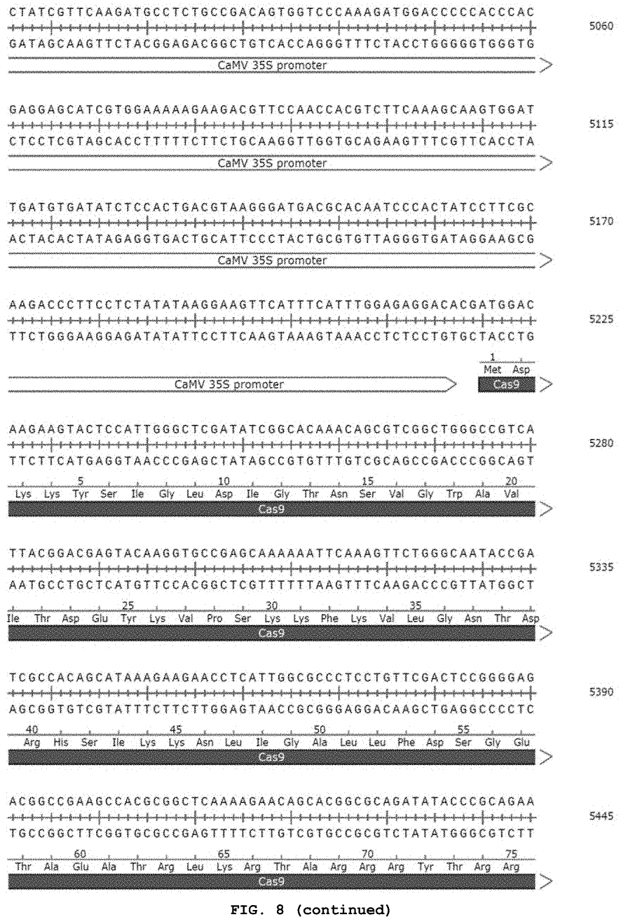

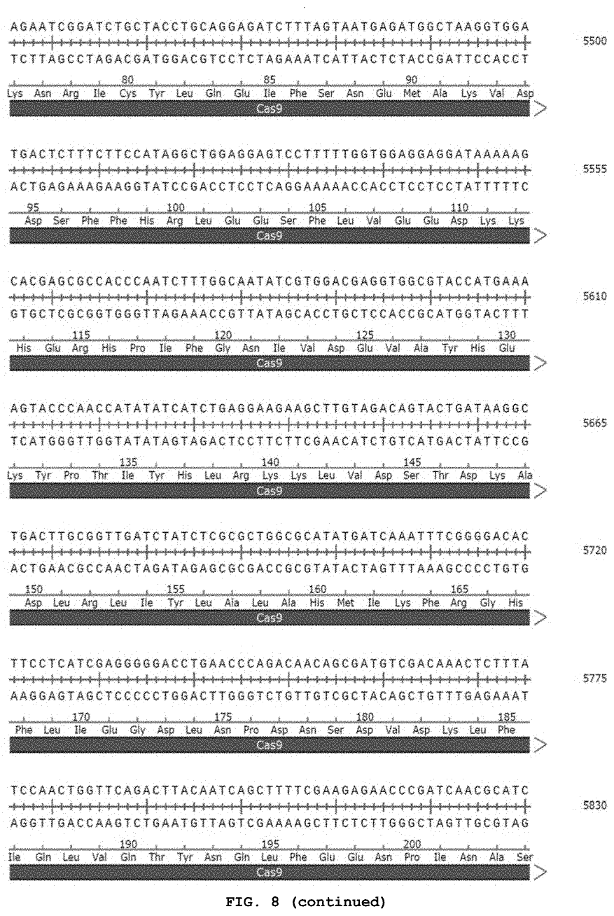

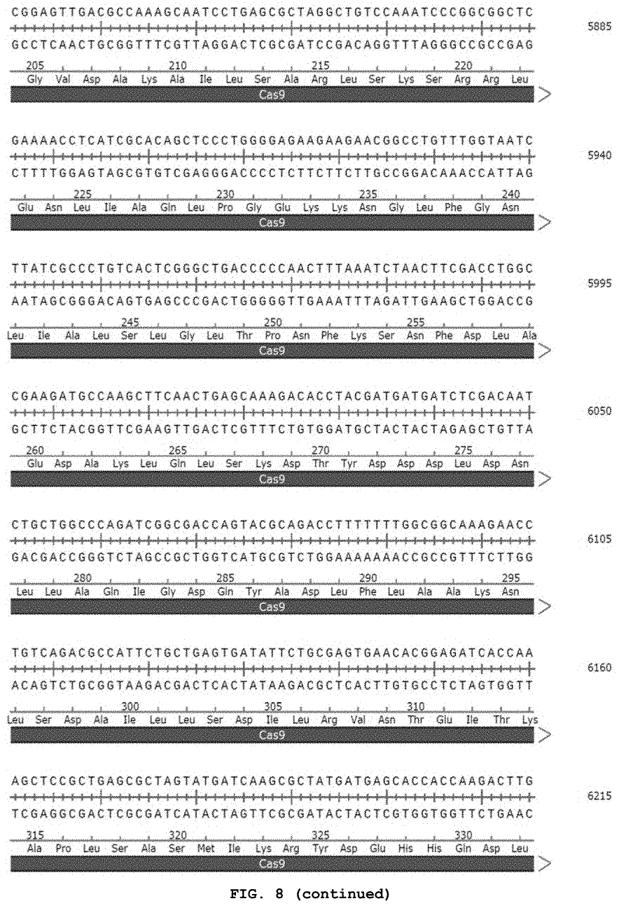

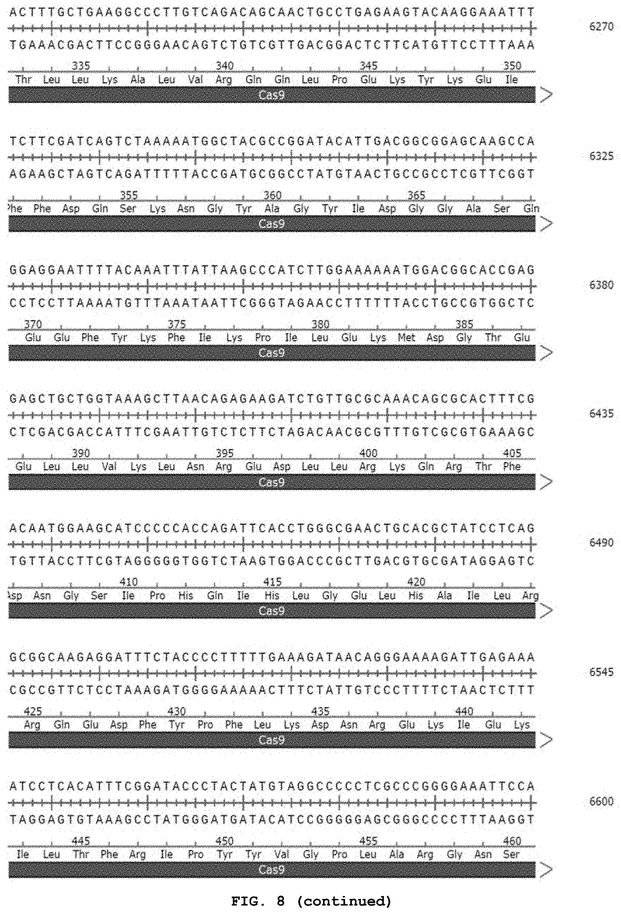

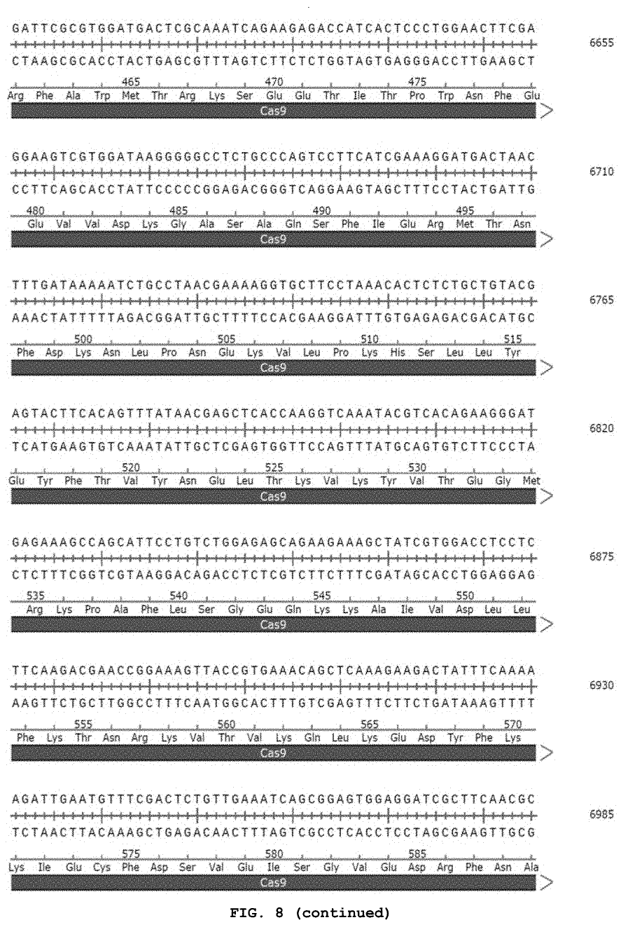

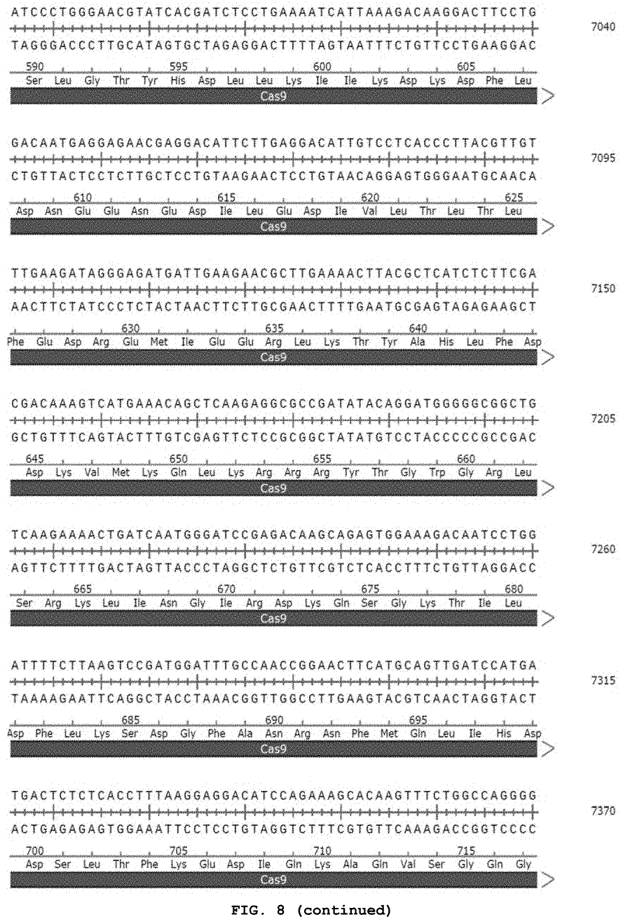

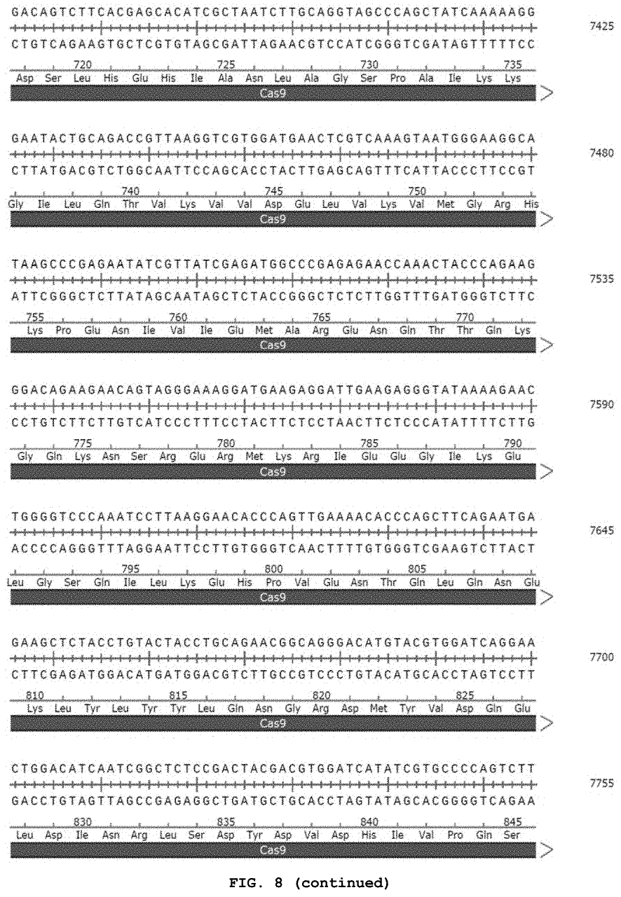

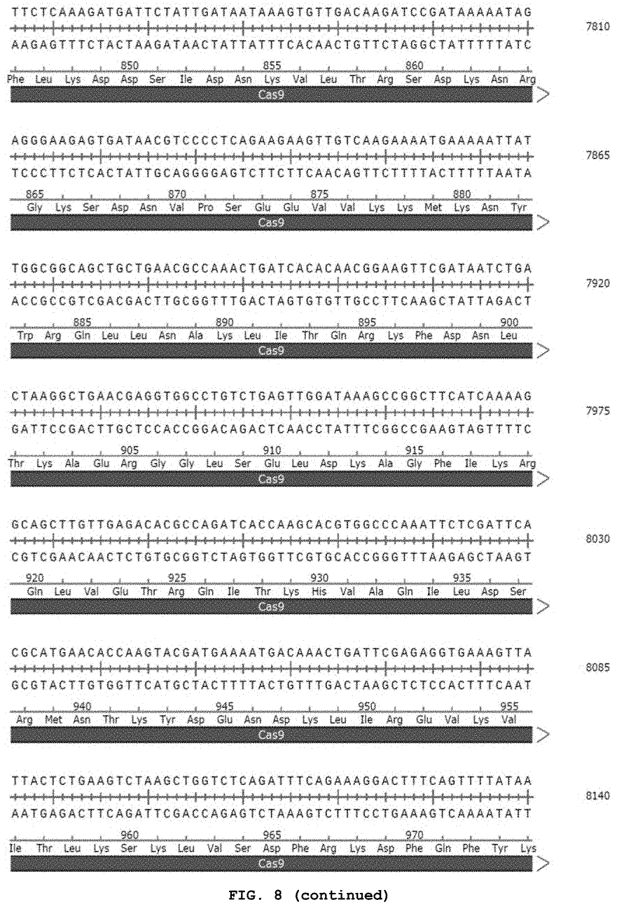

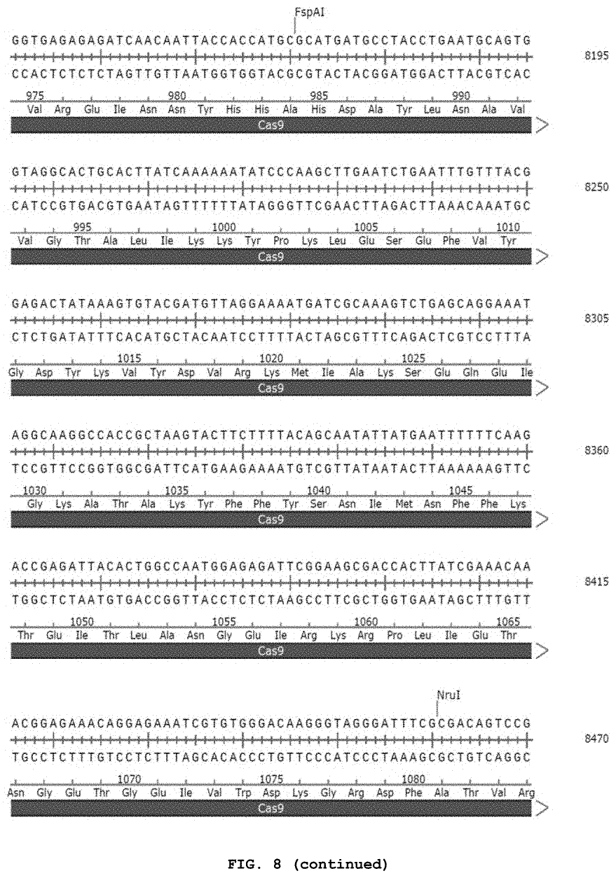

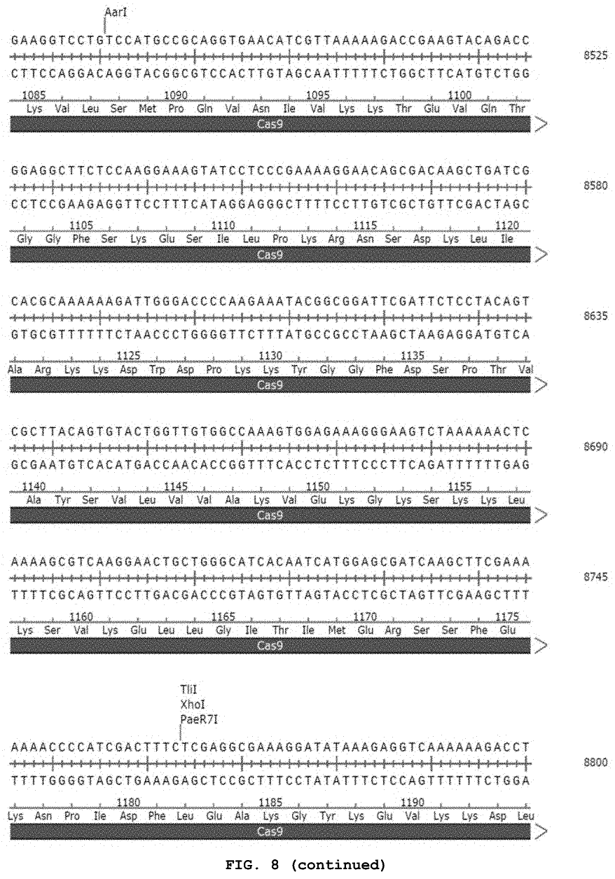

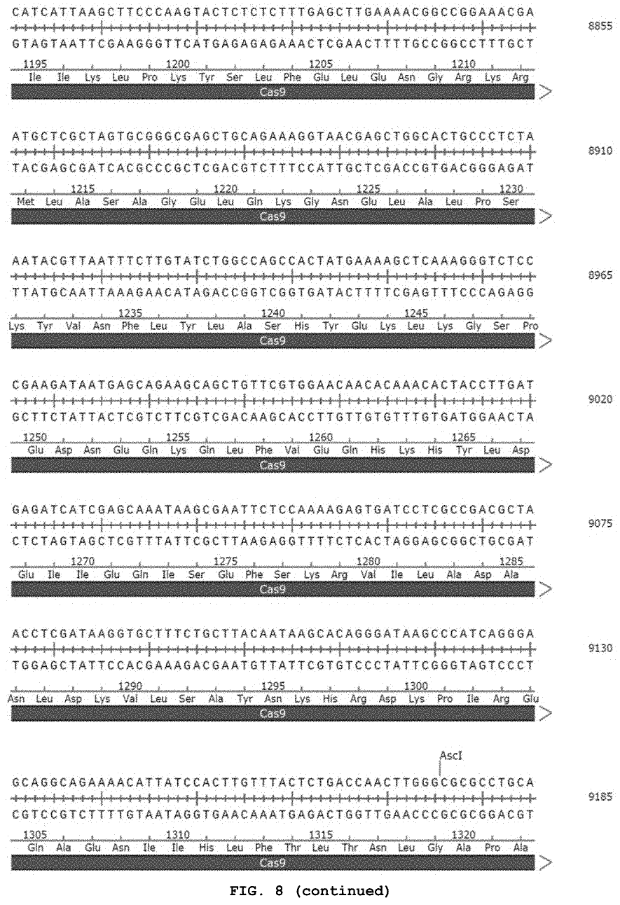

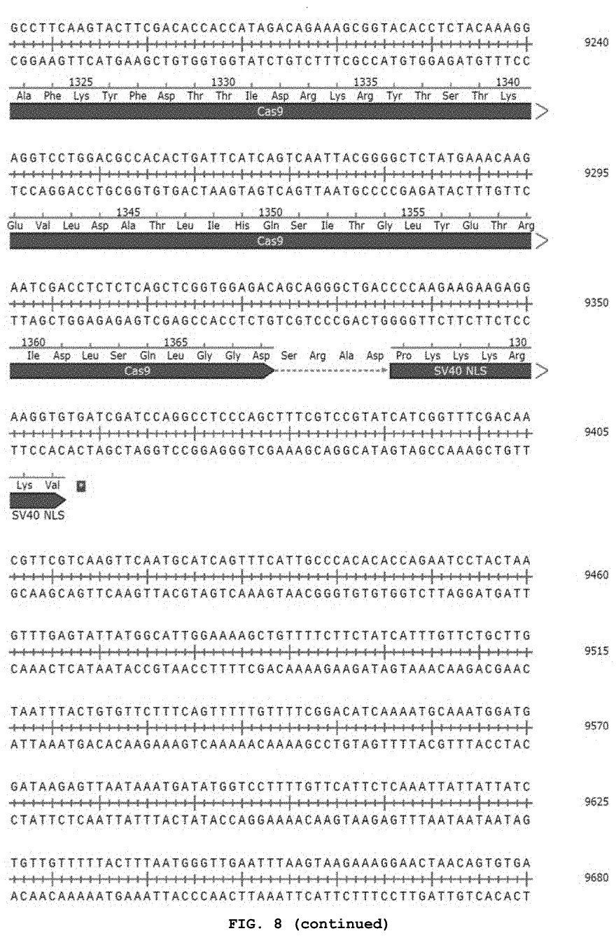

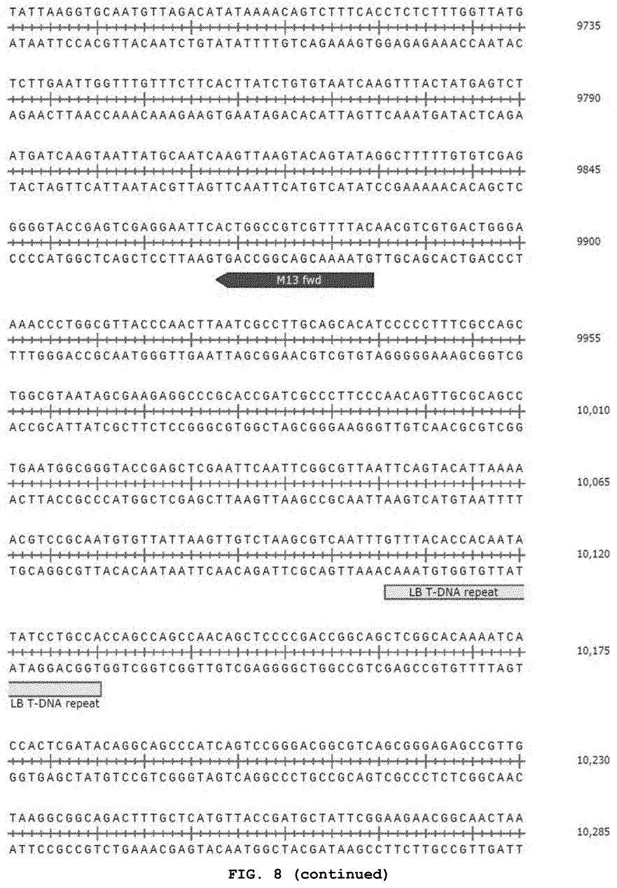

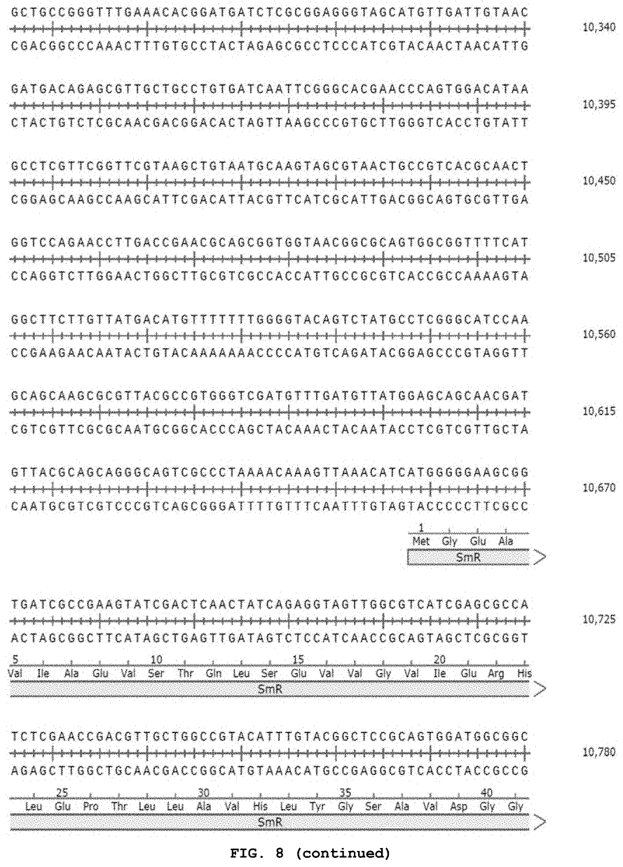

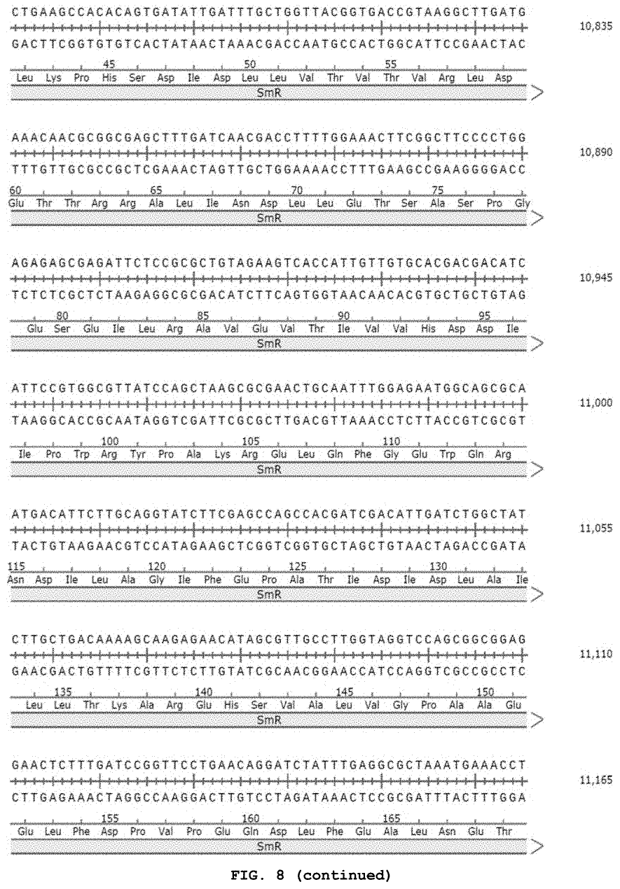

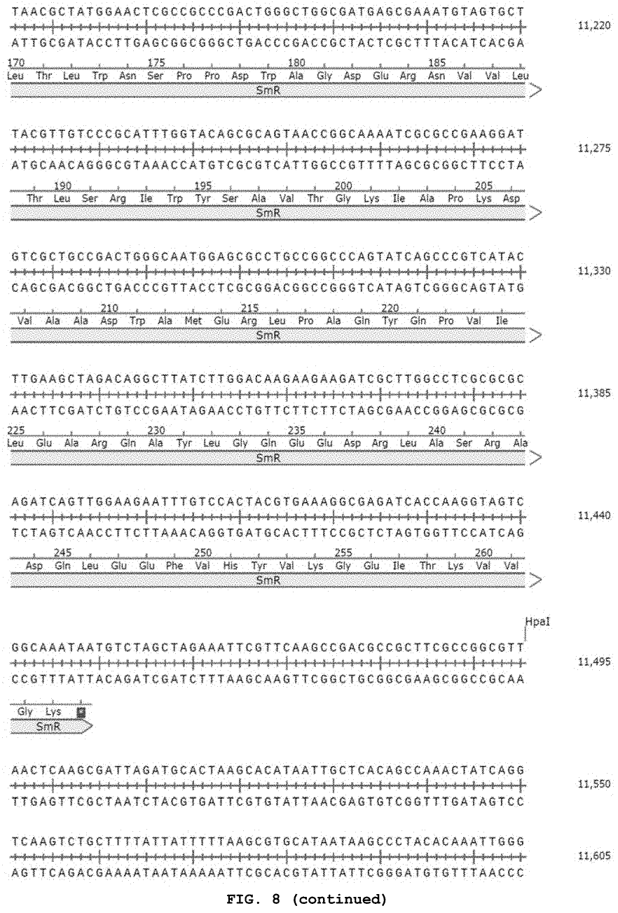

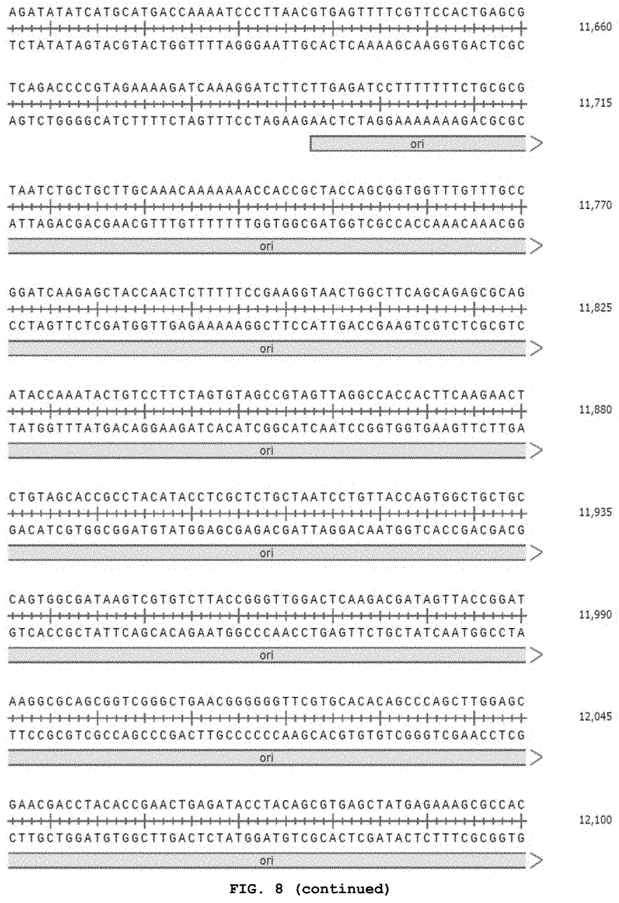

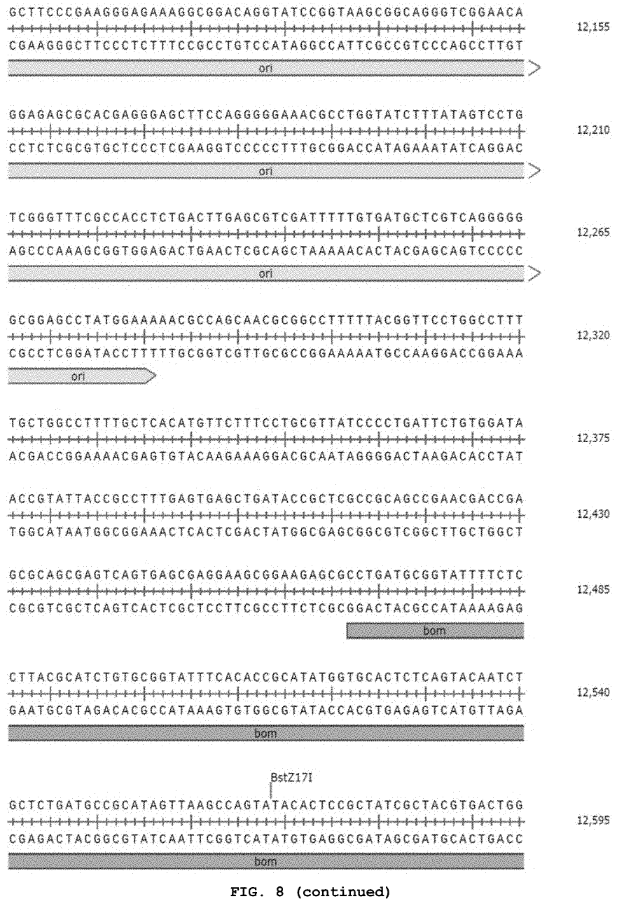

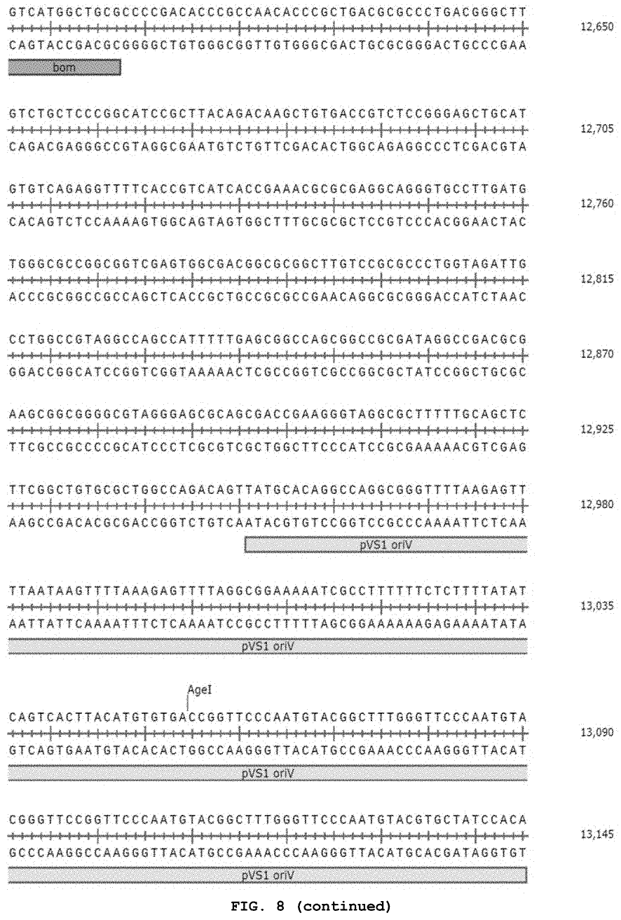

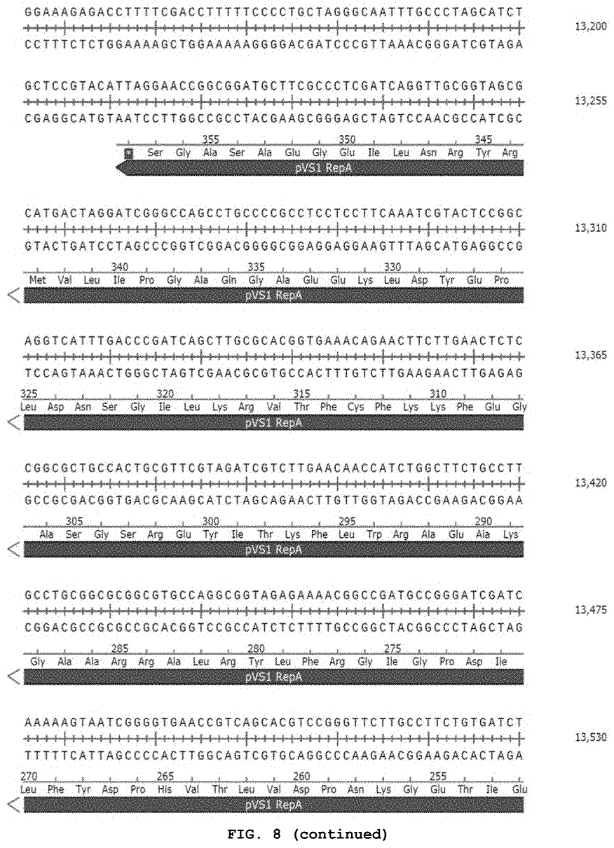

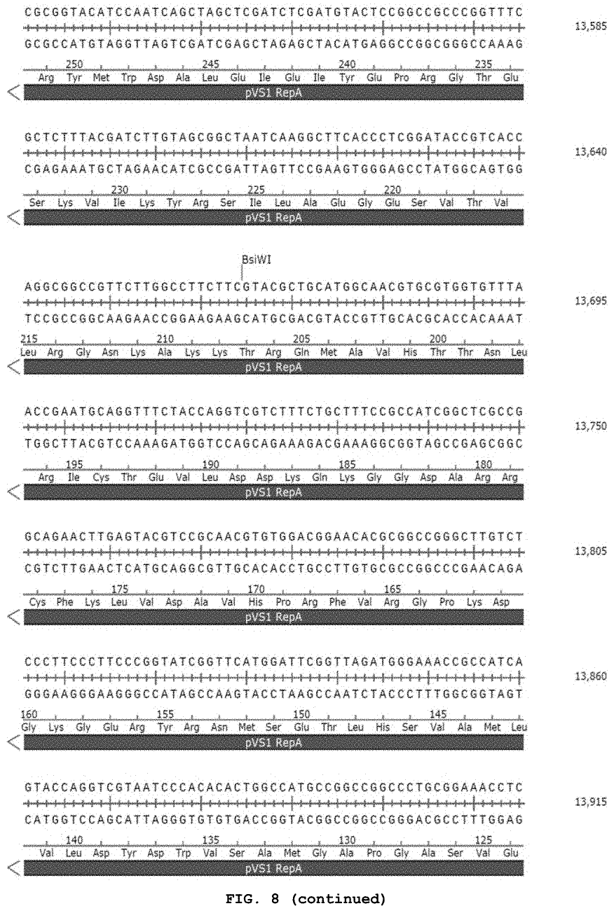

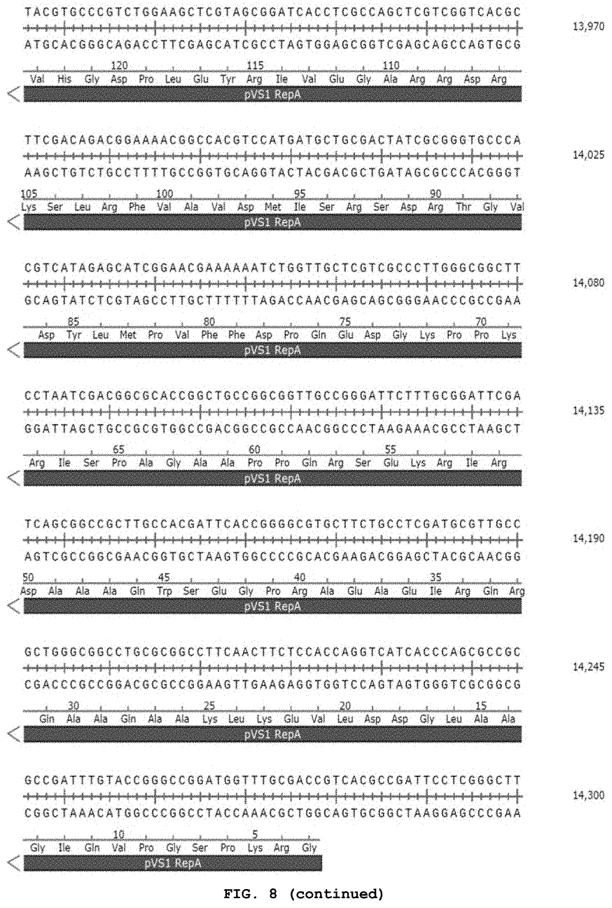

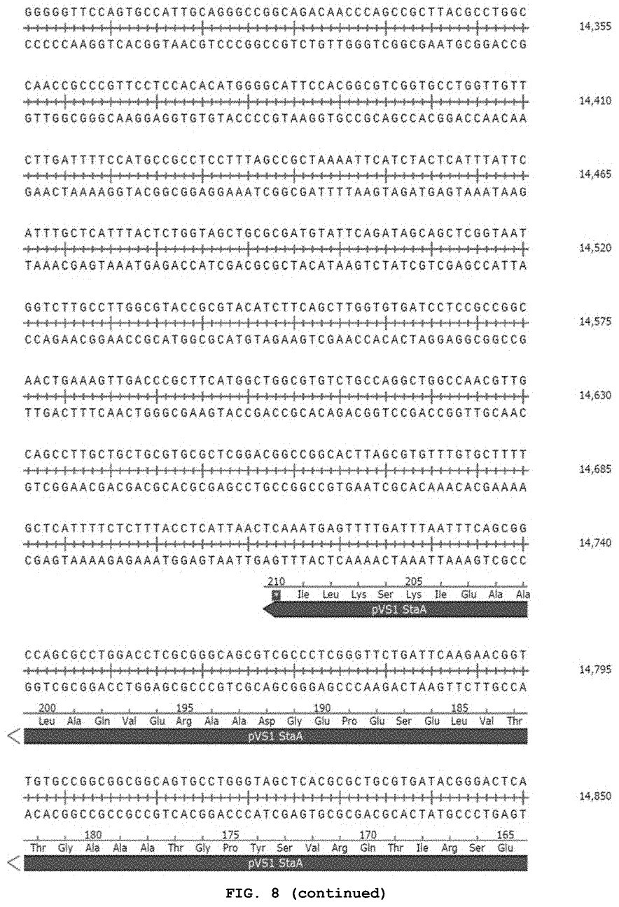

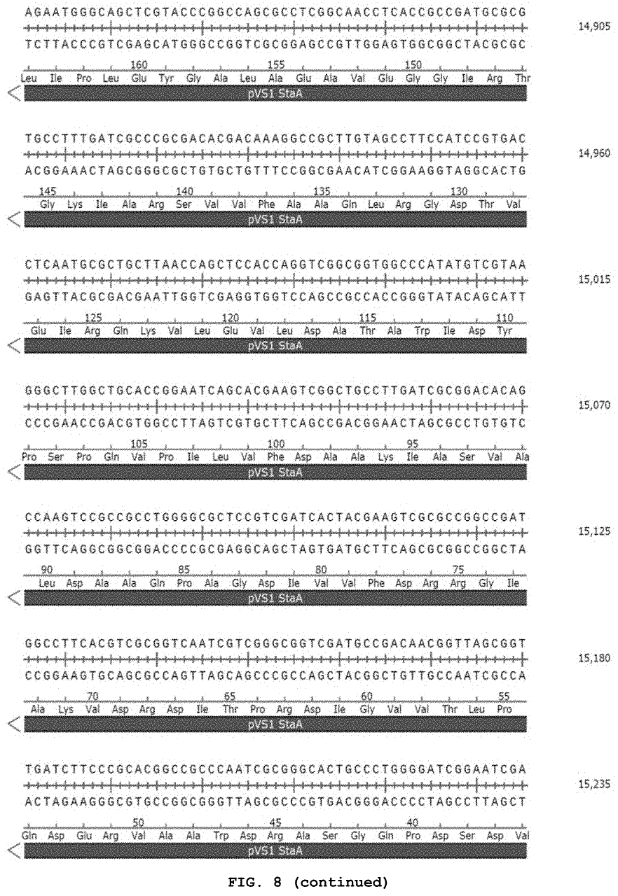

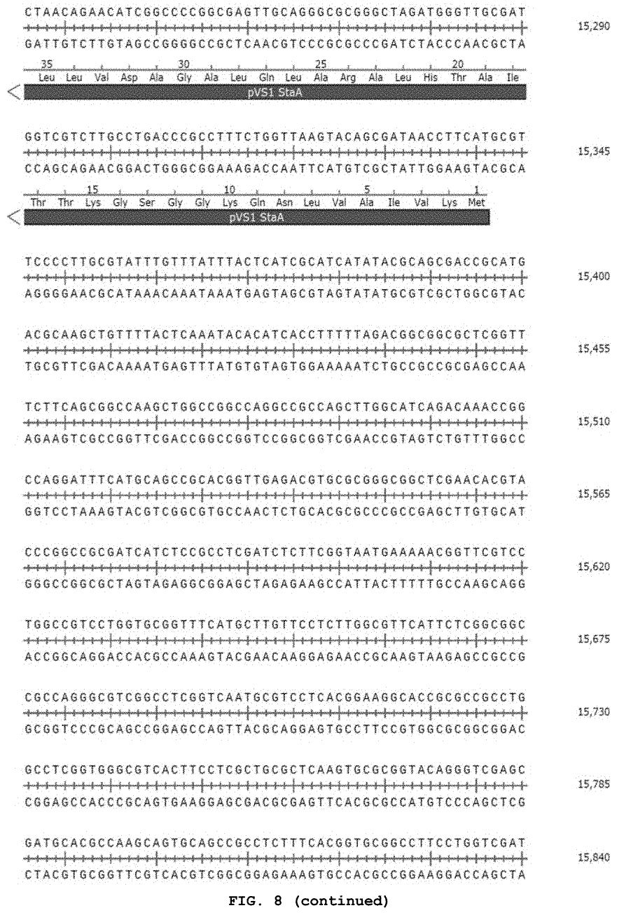

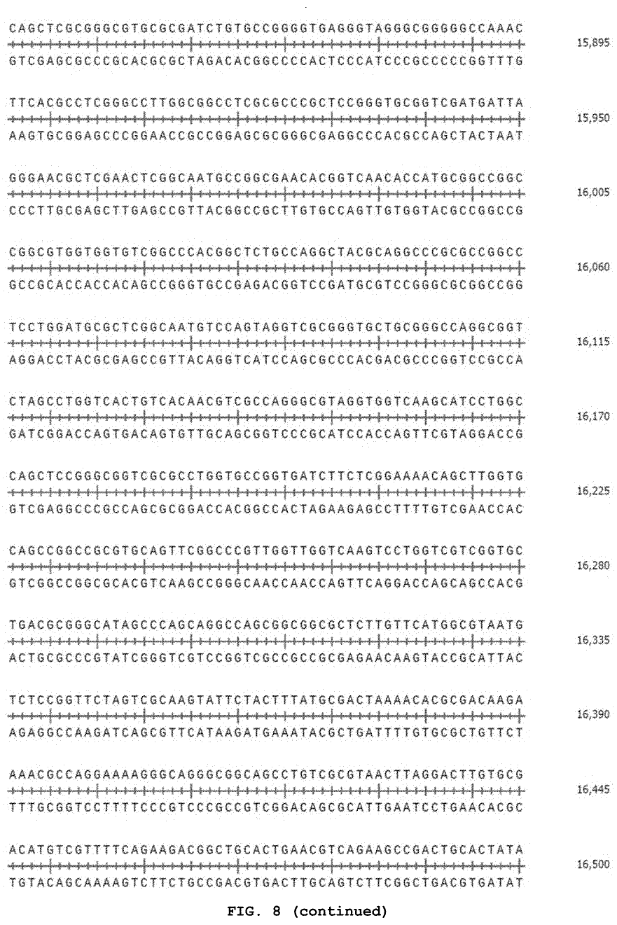

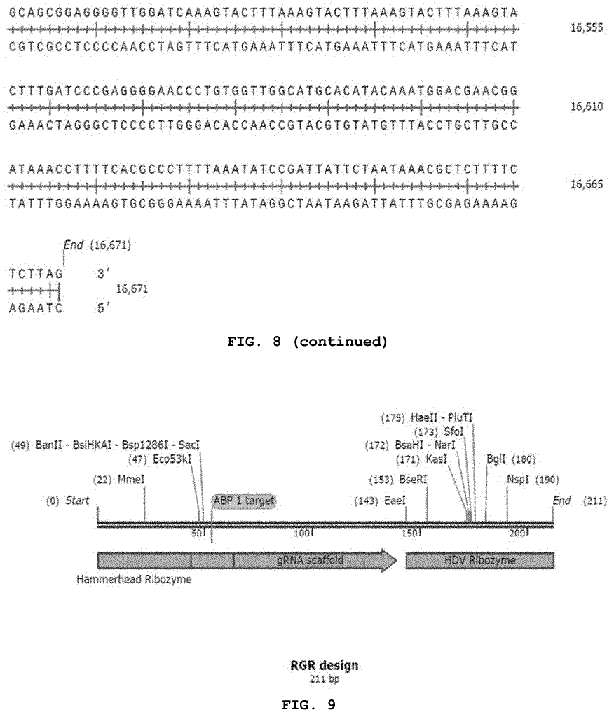

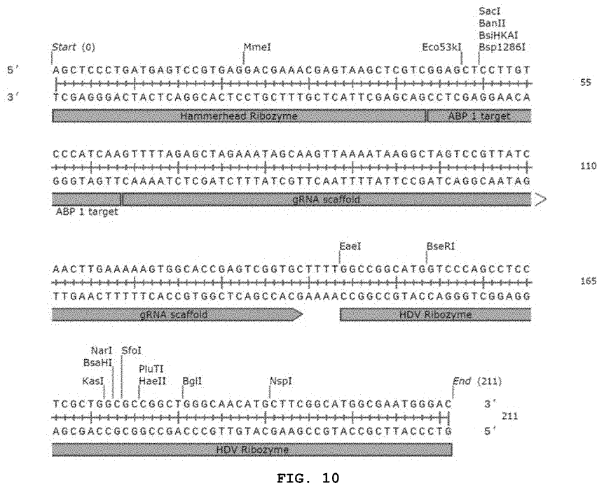

The disclosure demonstrates the use and design of a CRISPR/Cas9 vector to generate Cas9-free T2 plants with targeted mutations. The mutations in the Cas9-free plants are stable and are transmitted to next generations in a Mendelian fashion (see, Table II). The method is reliable and effective (see, FIG. 6A).

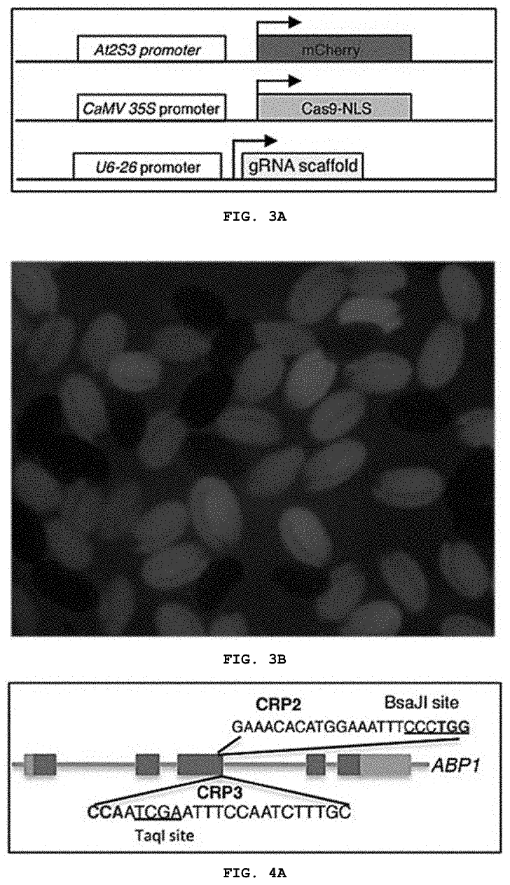

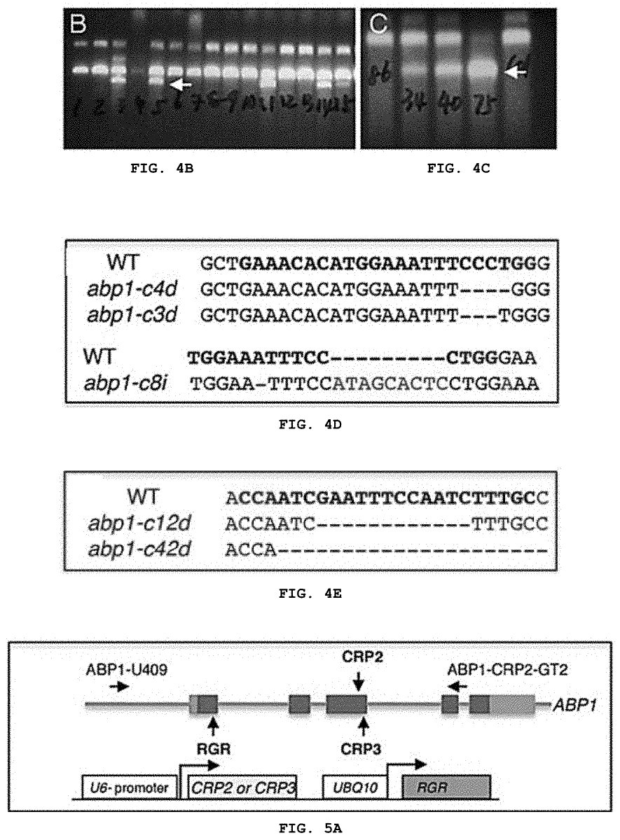

The disclosure shows that it is useful to focus on Cas9-free T2 plants in order to unambiguously identify heritable mutations generated by CRISPR/Cas9. Historically, the identification of a targeted mutation generated from CRISPR/Cas9 is mainly based on analyses of PCR fragments digested with enzymes. The PCR reactions usually use genomic DNA isolated from a piece of leaf tissue as a template. Results from such assays often cannot reveal the mosaic nature of the mutations if the majority of the cells contain the mutation (FIG. 4C), thus often yielding false positives. In addition, the chance for identifying a mutation in Cas9-free T2 plants is usually low (Table I). For example, the disclosure shows that only 1 plant that contained a heterozygous abp1 mutation (abp1-c8i) was identified after genotyping 95 Cas9-free T2 plants generated from the CRP2 T1 plant #25 (Table I and FIG. 4D). In order to identify one mutant plant in this case, one would have to genotype at least 380 T2 plants if one did not pre-select the Cas9-free plants. Given that less than 50% of the positive T1 plants produced Cas9-free plants with a mutation (Table I), the workload would be so heavy that identification of a heritable mutation in a Cas9-free plant becomes prohibitive if one does not pre-select the Cas9-free T2 plants. Expression of, for example, mCherry gene in seeds makes the selection of Cas9-free T2 plants very convenient and efficient (FIG. 3B).

A construct of the disclosure The disclosure provides recombinant engineered, non-naturally occurring gene editing system comprising one or more vectors comprising (a) at least one first regulatory element operable in a plant cell and operably linked to at least one nucleotide sequence encoding a CRISPR-Cas system guide RNA (gRNA) for targeting a target sequence in a plant, (b) a second regulatory element operable in a plant cell operably linked to a nucleotide sequence encoding a class 2 CRISPR-associated nuclease, and (c) a third regulatory element operable in a plant cell operably linked to a fluorescent reporter and optionally (d) a fourth regulatory element linked to an antibiotic resistance gene, wherein component (a) is located on the same or different vector than components (b) and (c), whereby the guide RNA targets the target sequence and the CRISPR-associated nuclease cleaves the DNA molecule, whereby expression of the at least one gene product is altered; and, wherein the CRISPR-associated nuclease and the guide RNA do not naturally occur together. In one embodiment, (a), (b), and/or (c) are operably linked to a terminator sequence functional in a plant cell. In one embodiment, the class 2 CRISPR-associated nuclease is Cas9.

The regulatory elements described herein refer to promoters. The promoter can be any promoter that drives transcription in a plant cell and includes minimal core promoter and strong constitutive promoters. For example, suitable promoters can be selected from the group consisting of AT2S3 (SEQ ID NO:17, from about nucleotide 81 to 494), NOS promoter (SEQ ID NO:17, from nucleotide 1960 to 2143), and CaMV 35S promoter (SEQ ID NO:17, from about 3643 to 3987). Other promoters and regulatory elements suitable for use in the constructs of the disclosure will be known in the art. Recently, it was reported that expression of Cas9 under the control of some specialty promoters could greatly increase gene-editing efficiency in Arabidopsis (Wang et al., 2015; Yan et al., 2015; Mao et al., 2016). It was reported that homozygous mutants could be obtained at T1 generation (Wang et al., 2015). However the studies did not determine what percentage of Cas9-free T2 plants contained the edited mutations. The disclosure contemplates that in addition to using a 35S promoter, other “specialty promoters” as described in Wan et al. 2015, Yan et al. 2015 and Mao et al. 2016) can be used instead of the 35S promoter to drive the mCherry cassette Cas9 unit to increase the efficiency for isolating Cas9-free heritable Arabidopsis mutations.

Where the promoter is upstream of a ribozyme of gRNA sequence, the promoter is typically a polIII promoter. For example, a first regulatory element can comprise a DNA-dependent RNA polymerase III (Pol III) promoter sequence. In a further embodiment, the Pol III promoter sequence is derived from a monocot plant. In still a further embodiment, the Pol III promoter comprises a U3 or U6 promoter sequence.

The constructs can include a number of different gRNA sequences directed to various genes of a plant. There are software programs available to those of skill in the art to generate gRNA sequences for use in CRISPR/Cas9 gene modifications. In one embodiment, the gRNA sequence is flanked by ribozyme sequences and comprises a general sequence as set forth in SEQ ID NO:22, wherein the “N”s correspond to the gRNA sequence (Ribozyme-gRNA-Ribozyme; also referred to herein as “RGR”). In still another embodiment, the gRNA sequence or an RGR is cloned into a site in a construct of SEQ ID NO:13 or 17.

As described above, the construct comprises a fluorescent report gene. Various reporter coding sequences which code for fluorescent proteins are known and that, when exposed to certain wavelengths of light, exhibit fluorescence. Examples of such proteins, include, but are not limited to, mCherry (see, e.g., SEQ ID NO:14), mOrange, GFP, EGFP, AmCyan1, AsRed2, mBanana, Dendra2, DSRed2, DsRed-Express, E2-Crimson, HcRed1, PAmCherry, mPlum, mRaspberry, mStrawberry, tdTomato, Timer, ZsGreen1, ZsYellow1, and YFP.

In one embodiment, the disclosure provides a polynucleotide comprising a gRNA sequence flanked by ribozymes. In another embodiment, the polynucleotide comprises a sequence as set forth in SEQ ID NO:22, or a sequence as set forth in SEQ ID NO:21, wherein nucleotides 44-139 are replaced with a desired gRNA sequence.

The disclosure also provides a polynucleotide construct of SEQ ID NO:13 or 17 or sequences that are at least 95% identical thereto. In still another embodiment, the disclosure provides a construct of SEQ ID NO:17, wherein nucleotides 301 to 319 comprise a RGR (e.g., SEQ ID NO:22).

Generation of a large deletion by employing two gRNAs can simplify the screening processes (see, FIG. 6A). No dramatic decrease in editing efficiency was observed when dual gRNAs were used (Table I). Another advantage is that large deletion mutations are more likely null compared to small deletion mutations.

The term “expression” with respect to a gene or polynucleotide refers to transcription of the gene or polynucleotide and, as appropriate, translation of the resulting mRNA transcript to a protein or polypeptide. Thus, as will be clear from the context, expression of a protein or polypeptide results from transcription and translation of the open reading frame.

The term “polynucleotide,” “nucleic acid” or “recombinant nucleic acid” refers to polynucleotides such as deoxyribonucleic acid (DNA), and, where appropriate, ribonucleic acid (RNA). It should be noted that polynucleotide sequences can encode functional nucleic acids (e.g., biologically active RNA such as siRNA etc.) or polypeptides. It will also be recognized by those of skill in the art that variations in a particular polynucleotide sequence that encodes a biologically active molecule or polypeptide can still produce the biologically active molecule. In other words, the sequences can vary without losing their function (e.g., a sequence that is at least 85%, 90%, 95%, 98%, or 99% identical to the sequences of this disclosure are encompassed by the disclosure). For example, due to the degeneracy of the genetic codes two different polynucleotide sequences can encode the same polypeptide sequence. Moreover, it should be noted that any sequences depicted herein can be double or single stranded. Where a single stranded sequence is depicted it should be noted that the complementary strand is also contemplated. Where a double stranded sequences is depicted each strand independently is also encompassed. In addition, various “domain” (i.e., defined function segments) may be present. This application includes sequences “consisting of”, “consisting essentially of” and “comprising” such segments. The disclosure also encompasses any sequence herein or any of the foregoing embodiments of this paragraph wherein T can be U or U can be T (i.e., DNA or RNA).

A “protein” or “polypeptide”, which terms are used interchangeably herein, comprises one or more chains of chemical building blocks called amino acids that are linked together by chemical bonds called peptide bonds. A protein or polypeptide can function as an enzyme.

The term “recombinant plant” refers to a plant (and progeny) that has been genetically modified to express or over-express endogenous polynucleotides, to delete or reduce expression of an endogenous polynucleotide, or to express non-endogenous sequences, such as those included in a vector.

Accordingly, the disclosure provides an “engineered” or “modified” plant that is produced via the introduction of genetic material into a host or parental plant of choice thereby modifying or altering the cellular physiology and biochemistry of the plant. Through the introduction of genetic material the parental host acquires new properties, e.g. the ability to produce a new, or greater quantities of, an intracellular metabolite or to reduce or eliminate the production of a particular trait or protein. Such plants are “engineered” to have improved or biologically different traits compared to the parental plant or wild-type plant.

An engineered or modified plant can also include in the alternative or in addition to the introduction of a genetic material into a host or parental host, the disruption, deletion or knocking out of a gene or polynucleotide to alter the cellular physiology and biochemistry of the plant. Through the reduction, disruption or knocking out of a gene or polynucleotide the plant acquires new or improved properties

A “native” or “wild-type” protein, enzyme, polynucleotide, gene, or cell, means a protein, enzyme, polynucleotide, gene, or cell that occurs in nature.

A “parental plant” refers to a plant used to generate a recombinant plant. The term “parental plant” describes, in one embodiment, a source plant, seed or cell that occurs in nature, e.g. a “wild-type” cell that has not been genetically modified. The term “parental plant” further describes a source plant, seed or cell that serves as the “parent” for further engineering. In this latter embodiment, the source plant, seed or cell may have been genetically engineered, but serves as a source for further genetic engineering.

Accordingly, a parental plant functions as a reference plant, seed or cell for successive genetic modification events. Each modification event can be accomplished by introducing one or more nucleic acid molecules in to the reference plant, seed or cell.

It is understood that a polynucleotide described herein include “genes” and that the nucleic acid molecules described above include “vectors” or “plasmids.” Accordingly, the term “gene”, also called a “structural gene” refers to a polynucleotide that codes for a particular polypeptide comprising a sequence of amino acids, which comprise all or part of one or more proteins or enzymes, and may include regulatory (non-transcribed) DNA sequences, such as promoter region or expression control elements, which determine, for example, the conditions under which the gene is expressed. The transcribed region of the gene may include untranslated regions, including introns, 5′-untranslated region (UTR), and 3′-UTR, as well as the coding sequence.

Those of skill in the art will recognize that, due to the degenerate nature of the genetic code, a variety of codons differing in their nucleotide sequences can be used to encode a given amino acid. A particular polynucleotide or gene sequence encoding a biosynthetic enzyme or polypeptide described above are referenced herein merely to illustrate an embodiment of the disclosure, and the disclosure includes polynucleotides of any sequence that encode a polypeptide comprising the same amino acid sequence of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate exemplary embodiments of the disclosure.

The disclosure provides polynucleotides in the form of recombinant DNA expression vectors or plasmids, as described in more detail elsewhere herein, that encode one or more target enzymes or molecules. Generally, such vectors can either replicate in the cytoplasm of the host or integrate into the chromosomal DNA of the host. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) form.

A polynucleotide of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

“Transformation” refers to the process by which a vector is introduced into a host cell, plant or organism. Transformation (or transduction, or transfection), can be achieved by any one of a number of means including electroporation, microinjection, biolistics (or particle bombardment-mediated delivery), or agrobacterium mediated transformation etc.

A “vector” generally refers to a polynucleotide that can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

The various components of an expression vector can vary widely, depending on the intended use of the vector and the host cell(s) in which the vector is intended to replicate or drive expression.

Thus, recombinant expression vectors contain at least one expression system, which, in turn, is composed of at least a portion of a gene coding sequences or desired sequence to be expressed operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

A protein or nucleic acid has “homology” or is “homologous” to a second protein or nucleic acid if the nucleic acid sequence that encodes the protein has a similar sequence to the nucleic acid sequence that encodes the second protein or if the two nucleic acid sequences encode proteins that have similar biological function. Alternatively, a protein or nucleic acid has homology to a second protein or nucleic acid if the two proteins or two nucleic acids have “similar” amino acid or nucleic acids sequences, respectively.

As used herein, two proteins or nucleic acids (or a regions thereof) are substantially homologous when the amino acid or nucleic acids sequences, as the case may be, have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

With further respect to plants, the polynucleotides and vectors described herein can be used to transform a number of monocotyledonous and dicotyledonous plants and plant cell systems, including dicots such as safflower, alfalfa, soybean, coffee, amaranth, rapeseed, peanut or sunflower, as well as monocots such as oil palm, sugarcane, banana, sudangrass, corn, wheat, rye, barley, oat, rice, millet, or sorghum. Also suitable are gymnosperms such as fir and pine.

Thus, the methods described herein can be utilized with dicotyledonous plants belonging, for example, to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. The methods described herein also can be utilized with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., Pinales, Ginkgoales, Cycadales and Gnetales.

The methods can be used over a broad range of plant species, including species from the dicot genera Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; the monocot genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, and Zea; or the gymnosperm genera Abies, Cunninghamia, Picea, Pinus, and Pseudotsuga.

A transformed cell, callus, tissue, or plant as described herein can be identified and isolated by selecting or screening the engineered cells for fluorescence or lack thereof and/or antibiotic resistance. In one embodiment, Cas9 containing cells/plants/seeds are identified by measuring fluorescence (e.g., mCherry) and Cas9-free cells/plants/seeds are identified by a lack of fluorescence. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. Polynucleotides that are stably incorporated into plant cells can be introduced into other plants using, for example, standard breeding techniques.

DNA constructs may be introduced into the genome of a desired plant host by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. For example, a DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment (see, e.g., Klein et al., Nature 327:70-73, 1987). Alternatively, a DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature (see, e.g., Horsch et al., Science 233:496-498, 1984, and Fraley et al., Proc. Nat'l. Acad. Sci. USA 80:4803, 1983). The virulence functions of the Agrobacterium tumefaciens host will direct the insertion of the construct and adjacent marker into the plant cell DNA when the cell is infected by the bacteria using binary T DNA vector (Bevan, Nuc. Acid Res. 12:8711-8721, 1984) or the co-cultivation procedure (Horsch et al., Science 227:1229-1231, 1985). Generally, the Agrobacterium transformation system is used to engineer dicotyledonous plants (Bevan et al., Ann. Rev. Genet 16:357-384, 1982; Rogers et al., Methods Enzymol. 118:627-641, 1986). The Agrobacterium transformation system may also be used to transform, as well as transfer, DNA to monocotyledonous plants and plant cells (see, e.g., Hernalsteen et al., EMBO J 3:3039-3041, 1984; Hooykass-Van Slogteren et al., Nature 311:763-764, 1984; Grimsley et al., Nature 325:1677-179, 1987; Boulton et al., Plant Mol. Biol. 12:31-40, 1989; and Gould et al., Plant Physiol. 95:426-434, 1991).

Alternative gene transfer and transformation methods include, but are not limited to, protoplast transformation through calcium-, polyethylene glycol (PEG)- or electroporation-mediated uptake of naked DNA (see Paszkowski et al., EMBO J3:2717-2722, 1984; Potrykus et al., Molec. Gen. Genet. 199:169-177, 1985; Fromm et al., Proc. Nat. Acad. Sci. USA 82:5824-5828, 1985; and Shimamoto, Nature 338:274-276, 1989) and electroporation of plant tissues (D'Halluin et al., Plant Cell 4:1495-1505, 1992). Additional methods for plant cell transformation include microinjection, silicon carbide mediated DNA uptake (Kaeppler et al., Plant Cell Reporter 9:415-418, 1990), and microprojectile bombardment (see, e.g., Klein et al., Proc. Nat. Acad. Sci. USA 85:4305-4309, 1988; and Gordon-Kamm et al., Plant Cell 2:603-618, 1990).

The disclosed methods and compositions can be used to insert exogenous sequences into a predetermined location in a plant cell genome. This is useful to provide expression of an introduced transgene into a plant genome which is dependent on its integration site. Accordingly, genes encoding, e.g., nutrients, antibiotics or therapeutic molecules can be inserted, by targeted recombination, into regions of a plant genome favorable to their expression. The compositions and methods can also be used to edit a mutant gene sequence at a precise location or to knockout a gene's function.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, which may use a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al., Ann. Rev. of Plant Phys. 38:467-486, 1987.

Nucleic acids introduced into a plant cell can be used to confer desired traits on essentially any plant. A wide variety of plants and plant cell systems may be engineered for the desired physiological and agronomic characteristics described herein using the nucleic acid constructs of the present disclosure and the various transformation methods mentioned above. In certain embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Thus, the disclosed methods and compositions have use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea. One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible marker genes (e.g., mCherry fluorescence) that may be present on the recombinant nucleic acid constructs. Such selection and screening methodologies are well known to those skilled in the art. In one embodiment, transformants are identified by using the marker, subsequent progeny are identified by genotyping/phenotyping and a lack of the marker thereby identifying stable transformants (see, FIG. 6).

The disclosure also encompasses seeds of the transgenic plants described above wherein the seed has the transgene or gene construct. The disclosure further encompasses the progeny, clones, cell lines or cells of the transgenic plants described above wherein said progeny, clone, cell line or cell has the transgene or gene construct.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”), each of which is incorporated herein by reference in its entirety.

Examples of protocols sufficient to direct persons of skill through in vitro amplification methods, including the polymerase chain reaction (PCR), the ligase chain reaction (LCR), QP-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13:563-564.

The invention is illustrated in the following examples, which are provided by way of illustration and are not intended to be limiting.

### EXAMPLES

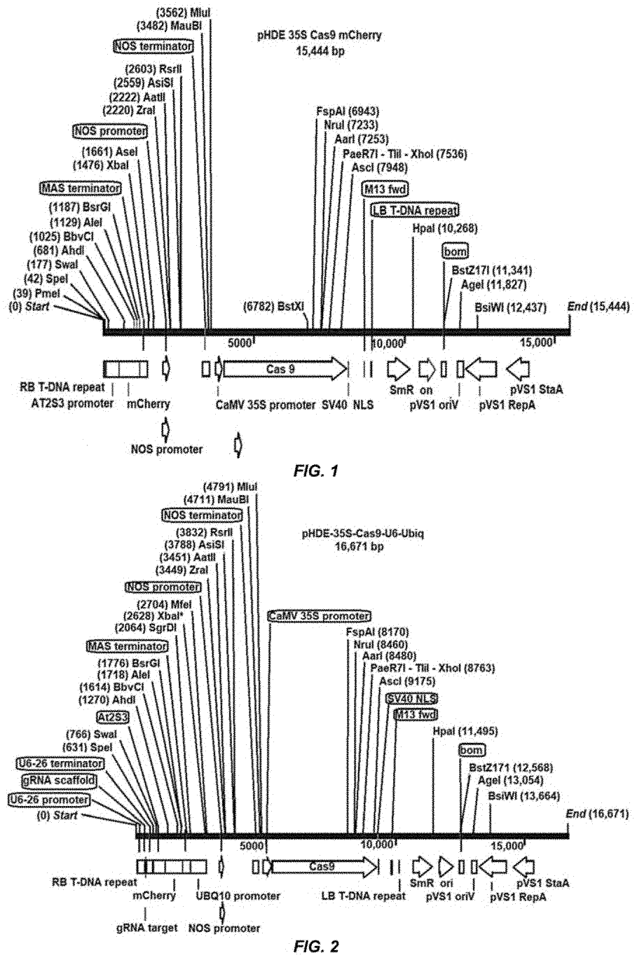

Two CRISPR/Cas9 vectors were designed and used: pHDE-35SCas9-mCherry and pHDE-35SCas9-UBQ10-mCherry. The maps and annotated vector sequences are shown in FIGS. 1 and 2. The U6-gRNA unit is cloned into the PmeI site in both vectors by Gibson Assembly (Gibson et al., 2008). The RGR unit was cloned into the MfeI site in pHDE-35SCas9-UBQ10-mCherry by Gibson Assembly. The RGR design and sequences are shown in FIG. 2.

The CRISPR/Cas9 constructs were transformed into Arabidopsis WT Col-0 through floral dipping. T1 plants were selected either by red fluorescence or on 16 μg/L hygromycin. Genomic DNA samples extracted from leaf tissues of two-week old T1 plants were used as templates for PCR reactions. To screen mutations at CRP2 target, the primer pair ABP1-U409 (CCTCATCACACAACAAAGTCACTC; SEQ ID NO:1) and ABP1-CRP2-GT2 (CATGAGGACCTGCAGGTGTTG; SEQ ID NO:2) were used to amplify the CRP2 target-containing fragment. The PCR product was digested using restriction enzyme BsaJI. Putative mutations should produce a BsaJI resistant band. To genotype mutations at the CRP3 site, primers ABP1-2E (TTGCCAATCGTGAGGAATATTAG; SEQ ID NO:3) and ABP1-CRP2-GT2 were used for PCR. Then digested the PCR product with TaqI. To screen for large deletions, PCR using ABP1-U409 and ABP1-CRP2-GT2 was conducted to screen for smaller fragments.

Cas9-free T2 seeds were isolated under a dissecting fluorescence microscope equipped with a mCherry filter. PCR screening was focused on mutants of the identified Cas9-free plants.

Development of a Visual Screen for Cas9-Free Plants.

In order to obtain stably transmissible mutations in Arabidopsis generated by CRISPR/Cas9-mediated genome editing technology, it is useful to segregate out the CRISPR/Cas9 construct. Otherwise, it is difficult to distinguish between a mutation transmitted from the previous generation and a newly generated mutation by Cas9. Traditionally, Cas9-free plants are identified by PCR using Cas9-specific primers. However, the PCR method is laborious and inefficient. In order to quickly identify Cas9-free plants, an mCherry expressing cassette was inserted into the CRISPR/Cas9 vector so that Cas9-free plants can be visually identified under a microscope (FIG. 1A). The mCherry gene was placed under the control of a strong promoter At2S3 (Kroj et al., 2003). As shown in FIG. 3B, seeds harvested from the T1 plants that contained the mCherry-expressing cassette segregate into two groups: one group displayed strong red fluorescence and the other group had no fluorescence. Because the mCherry cassette and the CRISPR/Cas9 unit are located on the same plasmid, a lack of red fluorescence is indicative of Cas9-free. Therefore, the mCherry cassette makes it very easy to visually differentiate the seeds with the Cas9 transgene from those without the Cas9 transgene (FIG. 3B).

Generation of mutations in the ABP1 locus by CRISPR/Cas9. An abp1 mutant that contains a 5 bp deletion in the first exon of ABP1 using a Ribozyme-based CRISPR technology was produced. The mutation in the first exon was suggested not to be optimal because of the potential production of truncated proteins (Chen et al., 2015; Dai et al., 2015; Habets and Offringa, 2015; Pan et al., 2015). Thus two new gRNAs were designed to target two discrete sites in the ABP1 gene (FIG. 4A) to test the vector and to generate additional abp1 alleles. The target sites (named CRP2 and CRP3) (FIG. 4A) were selected in an attempt to disrupt the auxin-binding pocket in ABP1. The CRP2 target has a BsaJI restriction site near the PAM motif and the CRP3 target contains a TaqI site (FIG. 4A). The two restriction enzymes can be used for screening editing events at the targets (FIG. 4B-C). The CaMV 35S promoter was used to drive the expression of Cas9 and used an U6 promoter to express the specific gRNAs (FIG. 3A). The CRISPR/Cas9 constructs were transformed into WT Arabidopsis Col-0 and screened for potential gene editing events in T1 plants.

As shown in Table I, T1 plants were identified that had undergone successful editing at the two ABP1 target sites. Interestingly, the editing efficiencies for the two target sites differed significantly. For CRP3 target, only 3.5% T1 plants (3/86) had detectable mutations at the target site. In contrast, the mutation rate was much higher at the CRP2 site. About 21% (7/33) T1 plants had detectable mutations at the CRP2 site. It was observed that the mutation rate at the CRP2 site was significantly underestimated because there are two overlapping BsaJI sites at the target. In addition, Cas9 usually cuts DNA 3 bp upstream of the PAM motif and mutations there would not disrupt the BsaJI restriction site at the CRP2 target. The exact reasons for why editing efficiencies varied greatly between the two targets are not fully understood. Recent studies have clearly shown that certain features in gRNAs greatly affected editing efficiency and that some guidelines for designing better gRNAs have been proposed (Chari et al., 2015; Liang et al., 2016). No apparent homozygous abp1 T1 plants were observed, even though the #75 plant appeared to contain very little WT ABP1 DNA at the CRP3 target site (FIG. 4C).

TABLE I
Editing efficiencies by CRISPR/Cas9 for different target sites

| Targets | CRP2 | CRP3 | CRP2/RGR | CRP3/RGR |
|---|---|---|---|---|
| T1 | 7/33 | 3/86 | 5/61 | 0/92 |
| T2 | T1#3, died | T1#34, 0/72 | T1#14, 0/48 | Not Analyzed |
| | T1#5, 0/72 | T1#40, 0/72 | T1#29, 26/52 (abp1-c2) | |
| | T1#11, 1/95 | T1#75, 8/196 | abp1-c2^{+/-} 13/52 | |
| | T1#14, 3/94 | abp1-c12d, 7/196 | abp1-c2^{-/-}, 13/52 | |
| | T1#25, 1/95 | abp1-c42d, 1/196 | T1#38, 0/72 | |
| | T1#30, 0/72 | | T1#56, 2/96 (abp1-c3) | |
| | T1#33, 0/96 | | T1#65, 0/72 | |

Note that the target sequences for CRP2 and CRP3 are described in FIG. 4A. RGR target site was previously described in [Gao Y. et al. (2015). Proc Natl Acad Sci USA 112: 2275-2280]. The RGR sequence and design are also shown in FIG. 2. CRP2/RGR refers to targeting both CRP2 and RGR sites simultaneously. The ratios represent the editing efficiency. For example, 7/33 refers to 7 positive plants out of 33 total plants. All of the T2 plants analyzed were Cas9-free based on a lack of red fluorescence (FIG. 3B). For the T2 plants, the number of mutant plants (both heterozygous and homozygous) from each T1 plant is shown. The abp1-c12d and abp1-c42d are from the same T1#75. The abp1-c2 shows the unusual segregation pattern.

Isolation of Cas9-Free and Stably Transmissible Abp1 mutations.

Seeds were harvested from individual T1 plants and used the fluorescence-based visual screen to identify T2 seeds that did not contain the CRISPR/Cas9 construct (FIG. 3B). The Cas9-free T2 seeds were then germinated and the seedlings transplanted to soil. At least 48 Cas9-free T2 plants harvested from each T1 plant were genotyped. Less than 50% (3/7) of the CRP2 T1 plants produced Cas9-free T2 plants with a mutation at the CRP2 target site (Table I). 95 Cas9-free T2 plants from the CRP2 T1 plant #11 were genotyped and 1 plant in this population was obtained that had a mutation (Table I), which was a 4 bp deletion (FIG. 4D) (abp1-c4d). The mutation was heterozygous. The 4 bp deletion resulted in a frame-shift. In theory, abp1-cod would produce a truncated ABP1 protein with the first 117 amino acid residues identical to those of WT ABP1. The abp1-cod is likely a loss-of-function mutant because it lacked the P(X)4H(X)3N fingerprint that is important for Zn and auxin binding (Woo et al., 2002). Among the 94 Cas9-free T2 plants from CRP2 T1 plant #14, 3 plants contained a 3 bp deletion, which were also heterozygous (abp1-c3d) (FIG. 4D). One heterozygous plant out of 95 Cas9-free T2 plants was identified from the CRP2 T1 plant #25 with a complex mutation pattern (abp1-c8i) (FIG. 4D). The abp1-c8i harbored a 9 bp insertion, a 1 bp deletion, and a point mutation (FIG. 4D). This allele will also be useful in future studies because of a lack of the key C-terminal region of ABP1.

To determine whether the mutations identified in the Cas9-free T2 plants could be stably transmitted to the next generations, 28 T3 plants generated from selfing a T2 abp1-c3d plant were genotyped. Thirteen plants were found to be heterozygous, 8 homozygous, and 7 without the mutation, indicating that the mutation identified in a Cas9-free plant at T2 stage was stably transmitted to the T3 generation in a Mendelian fashion (Table II). Genotyping results of the T3 plants from the other Cas9-free T2 mutants at the CRP2 targets were also consistent with the expected Mendelian ratios, suggesting that once a mutation is confirmed in a Cas9-free T2 plant, the mutation would be stable and could be transmitted to next generations following Mendelian genetics (Table II).

TABLE II
Segregation patterns of the various abp1 mutants generated by CRISPR/Cas9

| | Observed | Expected | |
|---|---|---|---|
| Mutant populations | (wt:abp1^{+/-}:abp1) | (wt:abp1^{+/-}:abp1) | Chi square |
| T3 from abp1-c4d^{+/-} (#11-238) | 7:11:6 | 1:2:1 | 0.25 |
| T3 from abp1-c3d^{+/-} (#14-132) | 7:13:8 | 1:2:1 | 0.214 |
| T3 from abp1-c8i^{+/-} (#25-417) | 9:13:6 | 1:2:1 | 0.786 |
| T3 from abp1-c12d (#75-23) | 8:17:9 | 1:2:1 | 0.059 |
| F1 of abp1-c12d (#75-43) X Col | 0:38:0 | 0:38:0 | 0 |
| F1 of abp1-c2^{-/-} (#29-56) X Col | 0:28:0 | 0:28:0 | 0 |
| T3 from abp1-c2^{+/-} (#29-41) | 19:46:17 | 1:2:1 | 0.452 |
| F1 of abp1-c3^{-/-} (#56-105) X Col | 0:53:0 | 0:53:0 | 0 |
| F1 of abp1-c3^{+/-} (#56-115) X Col | 26:22:0 | 24:24:0 | 0.333 |

Heredity of Various Adp1 Alleles was Analyzed Either a T3 Generation or F1 Plants.

The mutations generated at the CRP3 site were also analyzed. Among the 3 T1 plants that contained mutations at the CRP3 target, only one T1 plant produced Cas9-free offspring with mutations at the intended target site (Table I). Among the 196 Cas9-free T2 plants for CRP3 target genotyped, 8 plants contained mutations. Moreover, 7 out of the 8 plants had a 12 bp deletion at the CRP3 site and 1 contained a 42 bp deletion (FIG. 4E). Interestingly, among the plants with the 12 bp deletion (abp1-c12d), two were homozygous and 5 were heterozygous. The 42 bp deletion (abp1-c42d) was homozygous. The abp1-c12d homozygous T2 plants were crossed to WT plants and all of the F1 plants were heterozygous for the mutation (Table II). Some T3 plants from heterozygous T2 abp1-12d plants were genotyped. It was clear that the mutation segregated in Mendelian fashion (Table II).

Generation of Large Deletions Using Two gRNAs.

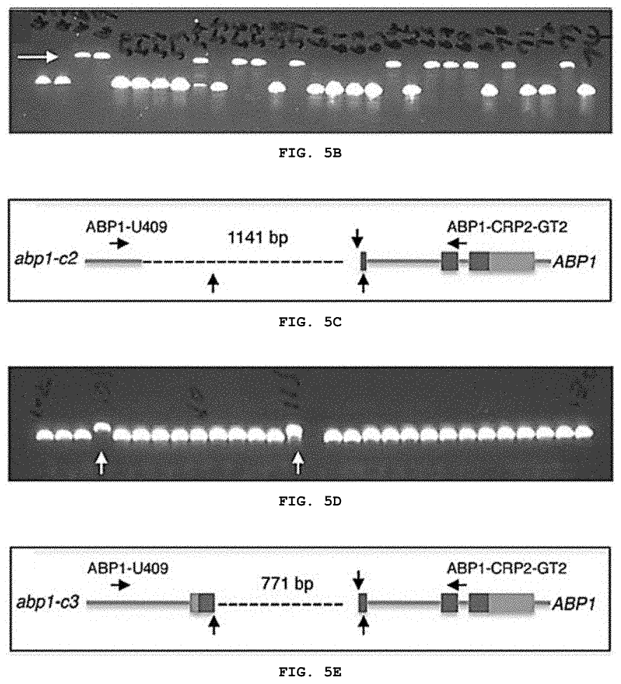

Experiments were performed to test whether a large fragment could be deleted by simultaneously expressing two gRNAs that target the sites flanking the intended deletion. If successful, such a strategy will greatly simplify the screening process for gene editing events because mutants will yield a much smaller PCR fragment than WT. Another advantage of a large deletion is that such a mutation would be an unambiguous knockout. A Ribozyme-flanked gRNA unit (RGR) that targeted the first exon of ABP1 successfully produced the abp1-c1 mutant with a 5 bp deletion (FIG. 5A). Here the same RGR unit was placed under the control of an Ubiquitin-10 promoter (UBQ10) to produce a gRNA targeting the first exon of ABP1 (FIG. 5A) (FIGS. 1 and 2 for vector map). Two dual-gRNA constructs were used to delete most of the genomic DNA of ABP1. The first construct combined the UBQ10:RGR unit with the U6:CRP2 and the other combined UBQ10:RGR with the U6:CRP3 (FIG. 5A). The two constructs were transformed into WT Arabidopsis and T1 plants were isolated. For the RGR/CRP3 construct, 92 T1 plants were genotyped, but none of them produced the expected small PCR fragment. Given the lower editing efficiency observed at the CRP3 site (Table I), the failure to generate a deletion from this construct was not a surprise. For the RGR/CRP2 construct, 5 T1 plants out of 61 produced a smaller PCR fragment than WT, suggesting that these two gRNAs together were able to cause the deletion of part of the ABP1 gene.

Cas9-free T2 plants from the T1 plants that were positive for deletions were screened to identify stably heritable abp1 deletion mutations. From the 5 positive T1 plants generated with RGR/CRP2, two T1 plants produced Cas9-free T2 offspring that contained a large deletion in the ABP1 gene. 52 Cas9-free T2 plants generated from a single T1 plant #29 were genotyped (FIG. 5B). 26 of the 52 T2 plants contained a large deletion at ABP1 locus and 13 of the mutants were homozygous for the mutation (Table I). The results apparently did not match the expected results from Mendelian genetics (Chi Square=19.5). The small PCR fragment were sequenced and found that the deletion was 1141 bp (abp1-c2) (FIG. 5C), which included part of the ABP1 promoter and the first three exons (FIG. 5C). Interestingly, the designed deletion between the two gRNAs was only 711 bp.

2 plants were identified at T2 generation that had a 711 bp deletion in the ABP1 locus (abp1-c3) after screening 96 Cas9-free progenies from the T1 plant #56 (FIG. 5D-E) (Table I). One plant was homozygous and the other was heterozygous. The deletion mutations identified in the T2 plants were tested to determine if they could be stably transmitted to next generations by genotyping T3 plants generated from selfing and by genotyping F1 plants resulted from a cross between the T2 mutants and WT. The results demonstrated that the two deletion mutants were stable and segregated into T3 plants following the rules of Mendelian genetics (Table II).

A number of embodiments of the invention have been described. Nevertheless, it will be understood that various modifications may be made without departing from the spirit and scope of the invention. Accordingly, other embodiments are within the scope of the following claims.

1. A recombinant engineered, non-naturally occurring gene editing system comprising one or more vectors comprising:
(a) at least one first regulatory element operable in a plant cell and operably linked to at least one nucleotide sequence encoding a CRISPR-Cas system guide RNA (gRNA) for targeting a target sequence in a plant,
(b) a second regulatory element operable in a plant cell operably linked to a nucleotide sequence encoding a class 2 CRISPR-associated nuclease, and
(c) a third regulatory element operable in a plant cell operably linked to a fluorescent reporter and
optionally (d) a fourth regulatory element linked to an antibiotic resistance gene,
wherein component (a) is located on the same or different vector than components (b) and (c), whereby the guide RNA targets the target sequence and the CRISPR-associated nuclease cleaves the DNA molecule, whereby expression of the at least one gene product is altered; and, wherein the CRISPR-associated nuclease and the guide RNA do not naturally occur together.

2. The recombinant engineered, non-naturally occurring gene editing system of claim 1 wherein (a), (b), and/or (c) are operably linked to a terminator sequence functional in a plant cell.

3. The recombinant engineered, non-naturally occurring gene editing system of claim 1 wherein said class 2 CRISPR-associated nuclease is Cas9.

4. The recombinant engineered, non-naturally occurring gene editing system of claim 1 wherein said plant is selected from the group consisting of Arabidopsis thaliana, Medicago truncatula, Solanum lycopersicum, Glycine max, Brachypodium distachyon, Oryza sativa, Sorghum bicolor, Zea mays, and Solanum tuberosum.

5. The recombinant engineered, non-naturally occurring gene editing system of claim 1, wherein the gRNA sequence is flanked by ribozyme sequences.

6. The recombinant engineered, non-naturally occurring gene editing system of claim 5, wherein the gRNA sequence has a general sequence of SEQ ID NO:22.

7. The recombinant engineered, non-naturally occurring gene editing system of claim 5, wherein the at least one first regulatory element comprises a pol II promoter sequence.

8. The recombinant engineered, non-naturally occurring gene editing system of claim 1 wherein said first regulatory element comprises a DNA-dependent RNA polymerase III (Pol III) promoter sequence.

9. The recombinant engineered, non-naturally occurring gene editing system of claim 8 wherein said Pol III promoter sequence is derived from a monocot plant.

10. The recombinant engineered, non-naturally occurring gene editing system of claim 7, wherein said Pol III promoter comprises a U3 or U6 promoter nucleotide sequence.

11. The recombinant engineered, non-naturally occurring gene editing system of claim 5, wherein the at least one first regulatory element is a UBQ10 promoter.

12. The recombinant engineered, non-naturally occurring gene editing system of claim 1, wherein the at least one first regulatory element comprises at least two regulatory elements separated by a gRNA sequence.

13. The recombinant engineered, non-naturally occurring gene editing system of claim 12, wherein the at least two regulatory element comprises a polIII promoter upstream of a gRNA sequence and a polIII promoter downstream of the gRNA sequence.

14. The recombinant engineered, non-naturally occurring gene editing system of claim 13, wherein the polIII promoter is upstream of a sequence of SEQ ID NO:22.

15. The recombinant engineered, non-naturally occurring gene editing system of claim 12, wherein the at least two regulatory elements comprise two pol III promoters.

16. The recombinant engineered, non-naturally occurring gene editing system of claim 12, having the structure U6 promoter—gRNA sequence—UBQ10 promoter—ribozyme—gRNA sequence—ribozyme.

17. The recombinant engineered, non-naturally occurring gene editing system of claim 1 wherein said second and/or third regulator element comprises a DNA-dependent RNA polymerase II (Pol II).

18. The recombinant engineered, non-naturally occurring gene editing system of claim 1, wherein the fluorescent reporter is selected from the group consisting of GFP (green fluorescent protein), EGFP (enhanced green fluorescent protein), GFP_{UV} (UV-excited green fluorescent protein), RFP (red fluorescent protein), mRFP (modified red fluorescent protein), YFP (yellow fluorescent protein), mcherry, CFP (cyan fluorescent protein), mGFP (modified green fluorescent protein), ERFP (enhanced red fluorescent protein), BFP (blue fluorescent protein), EBFP (enhanced blue fluorescent protein), EYFP (enhanced yellow fluorescent protein) and ECFP (enhanced cyan fluorescent protein).

19. The recombinant engineered, non-naturally occurring gene editing system of claim 18, wherein the fluorescent reporter is mCherry.

20. The recombinant engineered, non-naturally occurring gene editing system of claim 1 wherein the system is designed to alter the expression of the at least one gene product that confers one or more of the following traits: herbicide tolerance, drought tolerance, male sterility, insect resistance, abiotic stress tolerance, modified fatty acid metabolism, modified carbohydrate metabolism, modified seed yield, modified oil percent, modified protein percent, and resistance to bacterial disease, fungal disease or viral disease.

21. A modified plant cell produced by the recombinant engineered, non-naturally occurring gene editing system of claim 1.

22. A plant comprising the plant cell of claim 21.

23. A seed of the plant of claim 22.

24. A method comprising:
(a) transforming a plant with a recombinant engineered, non-naturally occurring gene editing system of claim 1;
(b) selecting T1 plants by fluorescence or antibiotic resistance;
(c) genotyping T1 plants to identify candidate plants and harvest seeds from individual plants;
(d) visually screening for class 2 CRISPR-associated nuclease-free T2 seeds by measuring fluorescence, wherein no fluorescence is indicative of class 2 CRISPR-associated nuclease-free seeds; and
(e) obtaining stable and heritable mutations from obtained T2 plants.

25. The method of claim 24 wherein the class 2 CRISPR-associated nuclease is Cas9.


SEQUENCE LISTING (22 sequences)

SEQ ID NO: 1 - DNA, 24 bp, Artificial Sequence
  Forward primer ABP1-U409
cctcatcacacaacaaagtcactc

SEQ ID NO: 2 - DNA, 21 bp, Artificial Sequence
  Reverse Primer ABP1-CRP2-GT2
catgaggacctgcaggtgttg

SEQ ID NO: 3 - DNA, 23 bp, Artificial Sequence
  Forward Primer ABP1-2E
ttgccaatcgtgaggaatattag

SEQ ID NO: 4 - DNA, 23 bp, Artificial Sequence
  CRP2 partial sequence wiht BsaJ1 site
gaaacacatggaaatttccctgg

SEQ ID NO: 5 - DNA, 23 bp, Artificial Sequence
  CRP3 partial sequence with TaqI site
ccaatcgaatttccaatctttgc

SEQ ID NO: 6 - DNA, 27 bp, Artificial Sequence
  Abp1 wild-type target site
gctgaaacacatggaaatttccctggg

SEQ ID NO: 7 - DNA, 23 bp, Artificial Sequence
  abp1-c4d mutant
gctgaaacacatggaaatttggg

SEQ ID NO: 8 - DNA, 24 bp, Artificial Sequence
  abp1-c3d mutant
gctgaaacacatggaaattttggg

SEQ ID NO: 9 - DNA, 18 bp, Artificial Sequence
  abp1 target sequence
tggaaatttccctgggaa

SEQ ID NO: 10 - DNA, 26 bp, Artificial Sequence
  abp1-c8i mutant
tggaatttccatagcactcctggaaa

SEQ ID NO: 11 - DNA, 25 bp, Artificial Sequence
  abp1 target sequence
accaatcgaatttccaatctttgcc

SEQ ID NO: 12 - DNA, 13 bp, Artificial Sequence
  abp1-c12d mutant
accaatctttgcc

SEQ ID NO: 13 - DNA, 15444 bp, Artificial Sequence
  pHDE-335SCas9-mCherry vector
  CDS (505)..(1197)
  CDS (3993)..(8132)
  CDS (9428)..(10222)
gtttacccgccaatatatcctgtcaaacactgatagtttaaactagtgaagtgaagcttggtctcaactatgagacctaagctggcacaactatatttccaacatcactagctaccatcaaaagattgacttctcatcttactcgattgaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacggtaccaacaatggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagagctcttggactcccatgttggcaaaggcaaccaaacaaacaatgaatgatccgctcctgcatatggggcggtttgagtatttcaactgccatttgggctgaattgaagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcgtattgagaccgaattctcgtctagacctaactgagtaagctagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactagatccccaaacaagcttgaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatccgctagaggatctcgacctgcaagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatataggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgggatcttggaggtgatgtaacatgatcacaagctgatcccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacagcttgcatgccagcttgggctgcaggtcgacgcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgatatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgatcgatccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctttttgtgtcgagggggtaccgagtcgaggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgggtaccgagctcgaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttaaagtactttaaagtactttaaagtactttgatcccgaggggaaccctgtggttggcatgcacatacaaatggacgaacggataaaccttttcacgcccttttaaatatccgattattctaataaacgctcttttctcttag

SEQ ID NO: 14 - protein, 230 aa, Artificial Sequence
  Synthetic Construct
MDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 15 - protein, 1379 aa, Artificial Sequence
  Synthetic Construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 16 - protein, 264 aa, Artificial Sequence
  Synthetic Construct
IMGEAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGKIAPKDVAADWAMERLPAQYQPVILEARQAYLGQEEDRLASRADQLEEFVHYVKGEITKVVGK

SEQ ID NO: 17 - DNA, 16671 bp, Artificial Sequence
  pHDE-35S-Cas9-mCherry-UBQ10 with U6 gRNA unit
  misc_feature (301)..(319): N represents any nucleotide sequence corresponding to a gRNA (guide RNA sequence)
  CDS (1094)..(1786)
  CDS (5220)..(9359)
  CDS (10658)..(11449)
gtttacccgccaatatatcctgtcaaacactgatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgnnnnnnnnnnnnnnnnnnngttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataactttgcttagatctaattcattccgttacacctctgatggaaactagtgaagtgaagcttggtctcaactatgagacctaagctggcacaactatatttccaacatcactagctaccatcaaaagattgacttctcatcttactcgattgaaaccaaattaacatagggtttttatttaaataaaagtttaaccttctttttaaaaaattgttcatagtgtcatgtcagaacaagagctacaaatcacacatagcatgcataagcggagctatgatgagtggtattgttttgttcgtcacttgtcactcttttccaacacataatcccgacaacaacgtaagagcatctctctctctccacacacactcatgcatgcatgcattcttacacgtgattgccatgcaaatctcctttctcacctataaatacaaaccaacccttcactacactcttcactcaaaccaaaacaagaaaacatacacaaatagcaaaacggtaccaacaatggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagagctcttggactcccatgttggcaaaggcaaccaaacaaacaatgaatgatccgctcctgcatatggggcggtttgagtatttcaactgccatttgggctgaattgaagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcgtattgagaccgaattctcgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagctcgcaattgacctaactgagtaagctagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactagatccccaaacaagcttgaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatccgctagaggatctcgacctgcaagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatataggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgggatcttggaggtgatgtaacatgatcacaagctgatcccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacagcttgcatgccagcttgggctgcaggtcgacgcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgatcgatccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctttttgtgtcgagggggtaccgagtcgaggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgggtaccgagctcgaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttaaagtactttaaagtactttaaagtactttgatcccgaggggaaccctgtggttggcatgcacatacaaatggacgaacggataaaccttttcacgcccttttaaatatccgattattctaataaacgctcttttctcttag

SEQ ID NO: 18 - protein, 230 aa, Artificial Sequence; identical to SEQ ID NO: 14
  Synthetic Construct

SEQ ID NO: 19 - protein, 1379 aa, Artificial Sequence; identical to SEQ ID NO: 15
  Synthetic Construct

SEQ ID NO: 20 - protein, 263 aa, Artificial Sequence
  Synthetic Construct
MGEAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGKIAPKDVAADWAMERLPAQYQPVILEARQAYLGQEEDRLASRADQLEEFVHYVKGEITKVVGK

SEQ ID NO: 21 - DNA, 211 bp, Artificial Sequence
  Ribozyme-Guide-Ribozyme (RGR) construct
agctccctgatgagtccgtgaggacgaaacgagtaagctcgtcggagctccttgtcccatcaagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggac

SEQ ID NO: 22 - DNA, 128 bp, Artificial Sequence
  generic RGR construct
  misc_feature (44)..(60): N is any sequence corresponding to a gRNA sequence for any gene
agctccctgatgagtccgtgaggacgaaacgagtaagctcgtcnnnnnnnnnnnnnnnnnggccggcatggtcccagcctcctcgctggcgccggctgggcaacatgcttcggcatggcgaatgggac